(19) United States
(12) Patent Application Publication
(10) Pub. No.: US 2020/0132897 A1
(43) Pub. Date: Apr. 30, 2020

(54) COSMETIC HOLOGRAPHIC WEARABLE OCULAR DEVICES AND METHODS OF PRODUCTION THEREOF

(71) Applicant: TruIris LLC, Columbus, OH (US)

(72) Inventors: Jefferson ODHNER, Amherst, NH (US); Robin SEARS, Columbus, OH (US)

(21) Appl. No.: 16/661,691

(22) Filed: Oct. 23, 2019

Related U.S. Application Data
(60) Provisional application No. 62/750,116, filed on Oct. 24, 2018.
(60) Provisional application No. 62/806,086, filed on Feb. 15, 2019.

(51) Int. Cl. G02B 5/18; G02C 7/04; A61F 2/16

(52) U.S. Cl. CPC G02B 5/1857; A61F 2/1613; G02C 7/046; G02B 5/1823

(57) ABSTRACT

Wearable ocular devices (such as ocular prostheses or contact lenses) utilizing diffraction gratings to produce color, as well as methods for producing such devices, are provided. A diffraction grating on the device may diffract the incident light to the observer. The result may be colored light that appears to originate from the wearer's eyes. The diffraction grating may achieve a look or feeling that is qualitatively or quantitatively different from the look or feeling achieved by previous devices that use dyes or inks.

### CROSS-REFERENCE

This application claims the benefit of U.S. Provisional Application Ser. No. 62/750,116, filed on Oct. 24, 2018, entitled “Cosmetic Holographic Contact Lenses and Methods of Production,” and U.S. Provisional Application Ser. No. 62/806,086, filed on Feb. 15, 2019, entitled “Cosmetic Holographic Wearable Ocular Devices and Methods of Production Thereof,” each of which is incorporated herein by reference in their entireties.

### BACKGROUND

Wearable ocular devices may find use in a number of applications, such as for correcting vision deficiencies. Prior wearable ocular devices, or methods of production thereof, may be less than desirable in some respects.

### SUMMARY

Previous techniques for imparting colors to wearable ocular devices often use dyes or inks. While a variety of colors can be made on wearable ocular devices using such techniques, the colors are produced through optical absorption and reflection. Inks and dyes absorb color from incident light and reflect light of a particular color to an observer.

Disclosed herein are wearable ocular devices utilizing diffraction gratings to produce color, as well as methods for producing such wearable ocular devices. A diffraction grating on the wearable ocular device may diffract the incident light to the observer. The result may be colored light that appears to originate from the wearer's eyes. The diffraction grating may achieve a look or feeling that is qualitatively or quantitatively different from the look or feeling achieved by previous wearable ocular devices that use dyes or inks.

In an aspect, a method of imparting a representation to a wearable ocular device may comprise: (a) applying an optically absorptive material to a surface of the device; (b) directing a first laser light along a first optical path to the surface of the device; (c) directing a second laser light along a second optical path to the surface of the device; and (d) creating an interference pattern between the first and second laser light at the surface of the device such that the optically absorptive material absorbs light at areas of constructive interference in the interference pattern and ablates nearby portions of the surface of the device, thereby imparting a diffraction grating to the surface of the device. The first and second laser light may be emitted by a single laser. The first and second laser light may be directed along the first and second optical paths, respectively, by a spatial filter. The first optical path may comprise a reference mirror and the second optical path may comprise an objective mirror. The first laser light may be directed from the reference mirror to a first portion of the surface of the device and the second laser light may be directed from the objective mirror to a second portion of the surface of the device. The first and second portions of the surface of the device may partially overlap. The first and second portions of the surface of the device may completely overlap. The method may further comprise repeating (a)-(d) to impart a plurality of diffraction gratings to the surface of the device. The representation may be an expression or designation. The expression or designation may be a geometric object. The geometric object may comprise a dot, a line, a quadrilateral, a rectangle, a square, a pentagon, a hexagon, a heptagon, an octagon, a nonagon, a decagon, an undecagon, a dodecagon, a polygon with more than 12 sides, an ellipse, an oval, or a circle. The expression or designation may provide an indication as to whether the device is properly centered or oriented on a wearer of the device. The expression or designation may be a repository of information about the device. The repository of information may comprise a barcode, a QR code, or a QR code with a circular hole in the center. The repository of information may be used to track the device during manufacturing or during an ophthalmological study or clinical trial. The expression or designation may be a character or term. The expression or designation may be an image. The image may comprise a symbol, a logo, a brand, a photograph, a work of art, or a cartoon. The image may be obtained through a scanning procedure. The expression or designation may be configured to alter an appearance of a wearer of the device for an artistic purpose. The expression or designation may be a color. The method may further comprise repeating (a)-(d) to impart first, second, and third diffraction gratings to the surface of the device. The first diffraction grating may impart a red hue to the device, the second diffraction grating may impart a green hue to the device, and the third diffraction grating may impart a blue hue to the device. The red, green, and blue hues may be chosen to impart a desired color to the device. The method may further comprise, prior to (a): (i) selecting a desired color to be imparted to the device, and (ii) determining optical parameters required to produce the first, second, and third diffraction gratings. The method may further comprise, prior to (a): (i) using an optical spectrometer or digital camera to determine a desired color to be imparted to the device, and (ii) determining optical parameters required to produce the first, second, and third diffraction gratings. The expression or designation may be an artificial pupil. The artificial pupil may comprise a moth eye structure. The method may further comprise removing the optically absorptive material from the surface of the device. The device may be a contact lens. The surface of the device may be a front surface of the contact lens. The surface of the device may be a back surface of the contact lens. The device may be an ocular prosthesis.

In another aspect, a method of imparting a representation to a wearable ocular device may comprise: (a) selecting a representation to be imparted to the device; (b) determining optical parameters required to produce a diffraction grating on a surface of the device that imparts the desired color to the device; (c) applying an optically absorptive material to the surface of the device; and (d) directing laser light along an optical path through the device to a mirror, such that a first portion of the laser light is reflected from the mirror and creates an interference pattern with a second portion of the laser light at the surface of the device, such that the optically absorptive material absorbs light at areas of constructive interference in the interference pattern and ablates nearby portions of the surface of the device, thereby imparting the diffraction grating to the surface of the device. The surface of the device may be configured such that a normal to the surface of the device makes an angle of at least 30 degrees with the laser light. The optical path may comprise a spatial filter. The method may further comprise repeating (a)-(d) to impart a plurality of diffraction gratings to the surface of the device. The representation may be an expression or designation. The expression or designation may be a geometric object. The geometric object may comprise a dot, a line, a quadrilateral, a rectangle, a square, a pentagon, a hexagon, a heptagon, an octagon, a nonagon, a decagon, an undecagon, a dodecagon, a polygon with more than 12 sides, an ellipse, an oval, or a circle. The expression or designation may provide an indication as to whether the device is properly centered or oriented on a wearer of the device. The expression or designation may be a repository of information about the device. The repository of information may comprise a barcode, a QR code, or a QR code with a circular hole in the center. The repository of information may be used to track the device during manufacturing or during an ophthalmological study or clinical trial. The expression or designation may be a character or term. The expression or designation may be an image. The image may comprise a symbol, a logo, a brand, a photograph, a work of art, or a cartoon. The image may be obtained through a scanning procedure. The expression or designation may be configured to alter an appearance of a wearer of the device for an artistic purpose. The expression or designation may be a color. The method may further comprise repeating (a)-(d) to impart first, second, and third diffraction gratings to the surface of the device. The first diffraction grating may impart a red hue to the device, the second diffraction grating may impart a green hue to the device, and the third diffraction grating may impart a blue hue to the device. The red, green, and blue hues may be chosen to impart a desired color to the device. The method may further comprise, prior to (a): (i) selecting a desired color to be imparted to the device, and (ii) determining optical parameters required to product the first, second, and third diffraction gratings. The method may further comprise, prior to (a): (i) using an optical spectrometer or digital camera to determine the desired color to be imparted to the device, and (ii) determining optical parameters required to produce the first, second, and third diffraction gratings. The expression or designation may be an artificial pupil. The artificial pupil may comprise a moth eye structure. The method may further comprise removing the optically absorptive material from the surface of the device. The device may be a contact lens. The surface of the device may be a front surface of the contact lens. The surface of the device may be a back surface of the contact lens. The device may be an ocular prosthesis.

In another aspect, a method of imparting a representation to a wearable ocular device may comprise: (a) applying a phase change material to a surface of the device; and (b) lithographically patterning the phase change material to impart a diffraction grating to the surface of the device. (a) may occur prior to (b). (b) may occur prior to (a). The method may further comprise repeating (a) and (b) to impart a plurality of diffraction gratings to the surface of the device. The representation may be an expression or designation. The expression or designation may be a geometric object. The geometric object may comprise a dot, a line, a quadrilateral, a rectangle, a square, a pentagon, a hexagon, a heptagon, an octagon, a nonagon, a decagon, an undecagon, a dodecagon, a polygon with more than 12 sides, an ellipse, an oval, or a circle. The expression or designation may provide an indication as to whether the device is properly centered or oriented on a wearer of the device. The expression or designation may be a repository of information about the device. The repository of information may comprise a barcode, a QR code, or a QR code with a circular hole in the center. The repository of information may be used to track the device during manufacturing or during an ophthalmological study or clinical trial. The expression or designation may be a character or term. The expression or designation may be an image. The image may comprise a symbol, a logo, a brand, a photograph, a work of art, or a cartoon. The image may be obtained through a scanning procedure. The expression or designation may be configured to alter an appearance of a wearer of the device for an artistic purpose. The expression or designation may be a color. The method may further comprise repeating (a) and (b) to impart first, second, and third diffraction gratings to the surface of the device. The first diffraction grating may impart a red hue to the device, the second diffraction grating may impart a green hue to the device, and the third diffraction grating may impart a blue hue to the device. The red, green, and blue hues may be chosen to impart a desired color to the device. The method may further comprise, prior to (a): (i) selecting a desired color to be imparted to the device, and (ii) determining optical parameters required to produce the first, second, and third diffraction gratings. The method may further comprise prior to (a): (i) using an optical spectrometer or digital camera to determine a desired color to be imparted to the device, and (ii) determining optical parameters required to produce the first, second, and third diffraction gratings. The expression or designation may be an artificial pupil. The artificial pupil may comprise a moth eye structure. The device may be a contact lens. The surface of the device may be a front surface of the contact lens. The surface of the device may be a back surface of the contact lens. The device may be an ocular prosthesis.

In another aspect, a method of imparting a representation to a wearable ocular device may comprise lithographically patterning a device comprising a material having a phase change material mixed therein, thereby imparting a diffraction grating to a surface of the device. The method may further comprise lithographically patterning the device a plurality of times, thereby imparting a plurality of diffraction gratings to the surface of the device. The representation may be an expression or designation. The expression or designation may be a geometric object. The geometric object may comprise a dot, a line, a quadrilateral, a rectangle, a square, a pentagon, a hexagon, a heptagon, an octagon, a nonagon, a decagon, an undecagon, a dodecagon, a polygon with more than 12 sides, an ellipse, an oval, or a circle. The expression or designation may provide an indication as to whether the device is properly centered or oriented on a wearer of the device. The expression or designation may be a repository of information about the device. The repository of information may comprise a barcode, a QR code, or a QR code with a circular hole in the center. The repository of information may be used to track the device during manufacturing or during an ophthalmological study or clinical trial. The expression or designation may be a character or term. The expression or designation may be an image. The image may comprise a symbol, a logo, a brand, a photograph, a work of art, or a cartoon. The image may be obtained through a scanning procedure. The expression or designation may be configured to alter an appearance of an eye of a wearer of the device for an artistic purpose. The expression or designation may be a color. The method may further comprise lithographically patterning the device three times, thereby imparting first, second, and third diffraction gratings to the surface of the device. The first diffraction grating may impart a red hue to the device, the second diffraction grating may impart a green hue to the device, and the third diffraction grating may impart a blue hue to the device. The red, green, and blue hues may be chosen to impart a desired color to the device. The method may further comprise (i) selecting a desired color to be imparted to the device, and (ii) determining optical parameters required to produce the first, second, and third diffraction gratings. The method may further comprise (i) using an optical spectrometer or digital camera to determine a desired color to be imparted to the device, and (ii) determining optical parameters required to produce the first, second, and third diffraction gratings. The expression or designation may be an artificial pupil. The artificial pupil may comprise a moth eye structure. The device may be a contact lens. The surface of the device may be a front surface of the contact lens. The surface of the device may be a back surface of the contact lens. The device may be an ocular prosthesis.

In another aspect, a method of imparting a representation to a wearable ocular device may comprise: (a) selecting the representation to be imparted to the device; (b) determining optical parameters required to produce a diffraction grating on a surface of the device that imparts the representation to the device; and (c) imprinting the diffraction grating on the surface of the device. The method may further comprise repeating (a)-(c) to impart a plurality of diffraction gratings to the surface of the device. The representation may be an expression or designation. The expression or designation may be a geometric object. The geometric object may comprise a dot, a line, a quadrilateral, a rectangle, a square, a pentagon, a hexagon, a heptagon, an octagon, a nonagon, a decagon, an undecagon, a dodecagon, a polygon with more than 12 sides, an ellipse, an oval, or a circle. The expression or designation may provide an indication as to whether the device is properly centered or oriented on a wearer of the device. The expression or designation may be a repository of information about the device. The repository of information may comprise a barcode, a QR code, or a QR code with a circular hole in the center. The repository of information may be used to track the device during manufacturing or during an ophthalmological study or clinical trial. The expression or designation may be a character or term. The expression or designation may be an image. The image may comprise a symbol, a logo, a brand, a photograph, a work of art, or a cartoon. The image may be obtained through a scanning procedure. The expression or designation may be configured to alter an appearance of a wearer of the device for an artistic purpose. The expression or designation may be a color. The method may further comprise repeating (a)-(c) to impart first, second, and third diffraction gratings to the surface of the device. The first diffraction grating may impart a red hue to the device, the second diffraction grating may impart a green hue to the device, and the third diffraction grating may impart a blue hue to the device. The red, green, and blue hues may be chosen to impart a desired color to the device. The method may further comprise (i) selecting a desired color to be imparted to the device, and (ii) determining optical parameters required to produce the first, second, and third diffraction gratings. The method may further comprise, prior to (a): (i) using an optical spectrometer or digital camera to determine the desired color to be imparted to the device, and (ii) determining optical parameters required to produce the first, second, and third diffraction gratings. The expression or designation may be an artificial pupil. The artificial pupil may comprise a moth eye structure. The device may comprise a contact lens. The surface of the device may be a front surface of the contact lens. The surface of the device may be a back surface of the contact lens.

In another aspect a colored wearable ocular device may comprise a diffraction grating applied to a surface of the device, the diffraction grating configured to impart a representation to the device. The diffraction grating may be imprinted on the surface of the device. The diffraction grating may comprise a plurality of regions that have been ablated from the surface of the device. The diffraction grating may comprise a lithographically patterned phase change material. The device may comprise a plurality of diffraction gratings applied to the surface of the device. The representation may be an expression or designation. The expression or designation may be a geometric object. The geometric object may comprise a dot, a line, a quadrilateral, a rectangle, a square, a pentagon, a hexagon, a heptagon, an octagon, a nonagon, a decagon, an undecagon, a dodecagon, a polygon with more than 12 sides, an ellipse, an oval, or a circle. The expression or designation may provide an indication as to whether the device is properly centered or oriented on a wearer of the device. The expression or designation may be a repository of information about the device. The repository of information may comprise a barcode, a QR code, or a QR code with a circular hole in the center. The repository of information may be used to track the device during manufacturing or during an ophthalmological study or clinical trial. The expression or designation may be a character or term. The expression or designation may be an image. The image may comprise a symbol, a logo, a brand, a photograph, a work of art, or a cartoon. The image may be obtained through a scanning procedure. The expression or designation may be configured to alter an appearance of a wearer of the device for an artistic purpose. The expression or designation may be a color. The device may comprise first, second, and third diffraction gratings applied to the surface of the device. The first diffraction grating may impart a red hue to the device, the second diffraction grating may impart a green hue to the device, and the third diffraction grating may impart a blue hue to the device. The red, green, and blue hues may be chosen to impart the desired color to the device. The expression or designation may be an artificial pupil. The artificial pupil may comprise a moth eye structure. The device may be a contact lens. The diffraction grating may be applied to a front surface of the contact lens. The diffraction grating may be applied to a back surface of the contact lens. The contact lens may be a soft contact lens. The contact lens may be a rigid gas permeable contact lens. The contact lens may be a hybrid contact lens. The device may be an ocular prosthesis.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A illustrates a front view of a wearable ocular device comprising a diffraction grating, in accordance with some embodiments;

FIG. 1B illustrates a side view of a wearable ocular device comprising a diffraction grating, in accordance with some embodiments;

FIG. 1C illustrates a first color chart of colors that may be imparted to a wearable ocular device using the systems and methods described herein;

FIG. 1D illustrates a second color chart of colors that may be imparted to a wearable ocular device using the systems and methods described herein;

FIG. 2A illustrates a flowchart for a method of imparting a representation to a wearable ocular device using transmission holography ablation to produce a diffraction grating on a surface of the device, in accordance with some embodiments;

FIG. 2B illustrates an optical setup for transmission holography ablation of a wearable ocular device, in accordance with some embodiments;

FIG. 3A illustrates a flowchart for a method of imparting a representation to a wearable ocular device using reflection holography ablation to produce a diffraction grating on a surface of the device, in accordance with some embodiments;

FIG. 3B illustrates an optical setup for reflection holography ablation of a wearable ocular device, in accordance with some embodiments;

FIG. 4 illustrates a flowchart for a method of imparting a representation to a wearable ocular device using a phase change material applied to a surface of the device to produce a diffraction grating on the surface of the device, in accordance with some embodiments;

FIG. 5 illustrates a flowchart for a method of imparting a representation to a wearable ocular device using a phase change material mixed into the device to produce a diffraction grating on a surface of the device, in accordance with some embodiments;

FIG. 6 illustrates a flowchart for a method of imparting a representation to a wearable ocular device using imprinting to produce a diffraction grating on a surface of the device, in accordance with some embodiments; and

FIG. 7 illustrates a computer system that is programmed or otherwise configured to operate any of the systems or methods described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, the term “wearable ocular device” may comprise any ocular device that may be worn by a user. For instance, a wearable ocular device may comprise a contact lens. A wearable ocular device may comprise bifocals. A wearable ocular device may comprise an ocular prosthesis.

Reference will now be made to the figures, wherein like numerals refer to like characters throughout. It will be appreciated that the figures are not necessarily drawn to scale.

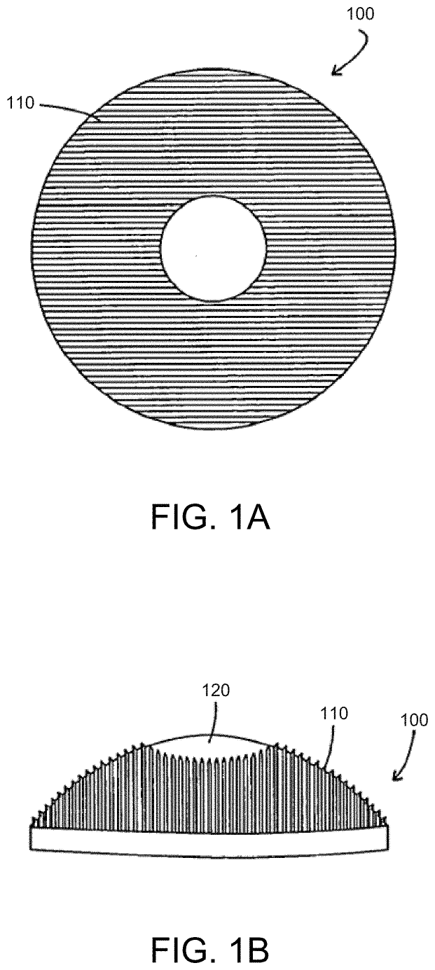

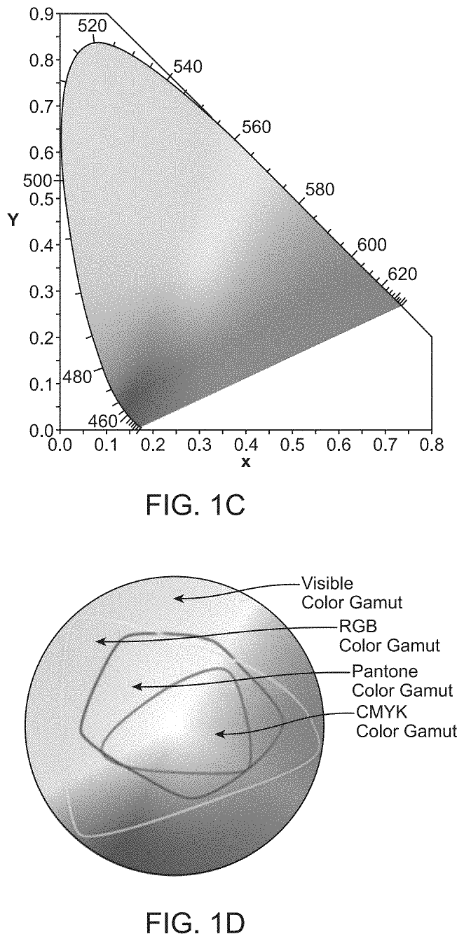

FIG. 1A shows a front view of a colored wearable ocular device 100 comprising a diffraction grating. As depicted in FIG. 1A, the device may comprise a contact lens. The contact lens may comprise a soft contact lens. The contact lens may comprise a disposable soft contact lens. The contact lens may comprise a soft daily contact lens. The contact lens may comprise a soft extended wear contact lens. The contact lens may comprise a hard contact lens. The contact lens may comprise a rigid gas permeable contact lens. The contact lens may comprise a hybrid contact lens. The contact lens may comprise a spherical lens. The contact lens may comprise a toric lens. The contact lens may comprise a monovision lens. The contact lens may comprise a bifocal lens. The contact lens may comprise a multifocal lens. Though depicted in FIG. 1A as a contact lens, the device 100 may comprise any wearable ocular device described herein. The device may comprise bifocals. The device may comprise an ocular prosthesis.

The ocular prosthesis may comprise an artificial eye. The ocular prosthesis may replace an absent natural eye. For instance, the ocular prosthesis may replace an absent natural eye following an enucleation, evisceration, orbital exenteration, or other removal of a natural eye. The ocular prosthesis may be shaped to fit under a user's eyelid. The ocular prosthesis may be shaped to fit over an orbital implant. The ocular prosthesis may comprise a convex shell shape. The ocular prosthesis may comprise a thin hard shell (e.g., a scleral shell) to be worn over a damaged eye. The ocular prosthesis may comprise a spherical shape. The ocular prosthesis may comprise a non-spherical shape. The ocular prosthesis may comprise a conical orbital implant (COI) or a multi-purpose conical orbital implant (MCOI). The ocular prosthesis may comprise a pyramid implant. The ocular prosthesis may comprise a flat surface. The ocular prosthesis may comprise preformed channels for rectus muscles of an eye. The ocular prosthesis may comprise a recessed slot for a superior rectus of an eye. The ocular prosthesis may comprise a protrusion to fill a superior fornix of an eye. The ocular prosthesis may comprise a conical shape that closely the anatomic shape of an ocular orbit. The ocular prosthesis may comprise a relatively wide anterior portion. The ocular prosthesis may comprise a relatively narrow posterior portion.

The ocular prosthesis may comprise a non-integrated implant. The ocular prosthesis may comprise a non-integrated spherical intraconal implant. The ocular prosthesis may comprise an integrated implant. The ocular prosthesis may comprise a quasi-integrated implant. The ocular prosthesis may comprise a coupling device. The ocular prosthesis may comprise a surface configured to improve implant motility of the ocular prosthesis. The ocular prosthesis may comprise an insert to accommodate a round-headed peg or screw. The round-headed peg or screw may transfer implant motility to the ocular prosthesis. The ocular prosthesis may be configured to allow for fibrovascular ingrowth following implantation of the ocular prosthesis.

The ocular prosthesis may comprise a glass eye. The ocular prosthesis may comprise a cryolite glass. The ocular prosthesis may comprise a sodium hexafluoroaluminate (Na₃AlF₆) glass. The ocular prosthesis may comprise a plastic. The ocular prosthesis may comprise a thermoplastic. The ocular prosthesis may comprise one or more materials selected from the group consisting of: polymethylmethacrylate (PMMA), hydroxyapatite (HA), polyethylene (PE), high density polyethylene, porous polyethylene (PP), high density porous polyethylene (Medpor), polyethylene terephthalate (PET), vicryl, silicone, and a bioceramic (such as aluminum oxide, Al₂O₃).

The device 100 may comprise a diffraction grating 110 applied to a surface of the device. The surface of the device may be a front surface of a contact lens. The surface of the device may be a back surface of a contact lens. The diffraction grating may be configured to impart a representation to the device. The representation may be an expression or a designation.

The expression or designation may be a geometric object. For instance, the diffraction grating may cause a viewer of the device to perceive one or more dots, lines, shapes (such as one or more triangles, quadrilaterals, rectangles, squares, pentagons, hexagons, heptagons, octagons, nonagons, decagons, undecagons, dodecagons, polygons with more than 12 sides, ellipses, ovals, circles, or any other geometric shape). Such markings may represent an indication of one or more optically relevant parameters of the device, such as whether the device is properly centered or oriented on a wearer of the device. In some cases, the markings may represent an indication of whether a contact lens is properly centered or oriented on an eye of a wearer of the contact lens. For instance, the marking may comprise a bump or lenticular that indicates an orientation of the contact lens.

The expression or designation may be a repository of information. For instance, the diffraction grating may cause a viewer of the device to perceiver a barcode, a QR code, or a QR code with a circular hole in its center. The repository of information may be useful for quality control or other tracking purposes. For instance, the repository of information may enable tracking of the device during manufacturing or during an ophthalmological study or clinical trial.

The expression or designation may be a character or term. The character or term may be a character or term selected from any language, such as Mandarin, Spanish, English, Hindi, Arabic, Portuguese, Bengali, Russian, Japanese, Punjabi, German, Javanese, Wu, Malay, Telugu, Vietnamese, Korean, French, Marathi, Tamil, Urdu, Turkish, Italian, Yue, Cantonese, Thai, Gujarati, Jin, Min, Persian, Polish, Pashto, Kannada, Xiang, Malayalam, Sundanese, Hausa, Odia, Burmese, Hakka, Ukrainian, Bhojpuri, Tagalog, Yoruba, Maithili, Uzbek, Sindhi, Amharic, Fula, Romanian, Oromo, Igbo, Azerbaijani, Awadhi, Gan, Cebuano, Dutch, Kurdish, Serbo-Croatian, Malagasy, Saraiki, Nepali, Sinhalese, Chittagonian, Zhuang, Khmer, Turkmen, Assamese, Madurese, Somali, Marwari, Magahi, Haryanvi, Hungarian, Chhattisgarhi, Greek, Chewa, Deccan, Akan, Kazakh, Sylheti, Zulu, Czech, Kinyarwanda, Dhundhari, Haitian, Creole, Ilocano, Quechua, Kirundi, Swedish, Hmong, Shona, Uyghur, Hiligaynon, Ilonggo, Mossi, Xhosa, Belarusian, Balochi, Konkani, or any other language.

The expression or designation may be an image, such as one or more logos, brands, photographs, works of art, cartoons, or other images. The image may be obtained through an image scanning procedure.

The expression or designation may be configured to alter an appearance of a wearer of the device for artistic purposes, such as for use in movies or other live action performances. In some cases, the expression or designation may be configured to alter an appearance of an eye of a wearer of a contact lens for artistic purposes. For instance, the expression or designation may alter the appearance of the wearer's eye such that the wearer appears to have the eyes of an animal, monster, or other non-human.

The expression or designation may be a color. In such a case, the diffraction grating may be configured to impart a desired color to the device. The diffraction grating may have the effect of taking light that strikes the diffraction grating and diffracting that light into multiple colors. The colors may be dispersed widely in angular space for a tightly spaced diffraction grating. The colors may be dispersed narrowly in angular space for a less tightly spaced diffraction grating. An observer who views the device may perceive the color of the device as a color of the rainbow which depends on the observer's viewing angle and the angle from which illumination light strikes the diffraction grating.

The expression or designation may be an artificial pupil. The artificial pupil may comprise one or more moth eye structures.

The diffraction grating may be designed using a variety of optical parameters, such as how tightly the diffraction grating is spaced. By careful selection of the optical parameters, the diffraction grating may be designed such that an observer perceives a rainbow of colors or the observer perceives a single color over a wide angle. The diffraction grating may be a simple grating, a compound grating, a blazed grating, or a pattern of grating dots.

FIG. 1B shows a side view of a colored wearable ocular device 100 comprising a diffraction grating. As shown in FIG. 1B, the device may be designed such that a wearer of the device does not perceive a change in the wearer's vision due to the presence of the diffraction grating 110. The diffraction grating may be annular in shape so as to leave a transparent region 120 of the device over a wearer's iris, allowing light to pass through a lens of a wearer's eye and strike the wearer's retina.

The device 100 may comprise a plurality of diffraction gratings applied to the surface of the device. The device may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, or more diffraction gratings applied to the surface of the device. The device may comprise at most 100, at most 90, at most 80, at most 70, at most 60, at most 50, at most 40, at most 30, at most 20, at most 19, at most 18, at most 17, at most 16, at most 15, at most 14, at most 13, at most 12 at most 11, at most 10, at most 9, at most 8, at most 7, at most 6, at most 5, at most 4, at most 3, at most 2, or fewer diffraction gratings applied to the surface of the device. The device may comprise a number of diffraction gratings that is within a range defined by any two of the preceding values applied to the surface of the device. Any two or more of the diffraction gratings may be arranged at any angle to one another. For instance, any two or more of the diffraction gratings may be arranged at an angle of at least 1 degree, at least 2 degrees, at least 3 degrees, at least 4 degrees, at least 5 degrees, at least 6 degrees, at least 7 degrees, at least 8 degrees, at least 9 degrees, at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 81 degrees, at least 82 degrees, at least 83 degrees, at least 84 degrees, at least 85 degrees, at least 86 degrees, at least 87 degrees, at least 88 degrees, at least 89 degrees, or more, to one another. Any two or more of the diffraction gratings may be arranged at an angle of at most 90 degrees, at most 89 degrees, at most 88 degrees, at most 87 degrees, at most 86 degrees, at most 85 degrees, at most 84 degrees, at most 83 degrees, at most 82 degrees, at most 81 degrees, at most 80 degrees, at most 75 degrees, at most 70 degrees, at most 65 degrees, at most 60 degrees, at most 55 degrees, at most 50 degrees, at most 45 degrees, at most 40 degrees, at most 35 degrees, at most 30 degrees, at most 25 degrees, at most 20 degrees, at most 15 degrees, at most 10 degrees, at most 9 degrees, at most 8 degrees, at most 7 degrees, at most 6 degrees, at most 5 degrees, at most 4 degrees, at most 3 degrees, at most 2 degrees, at most 1 degrees, or less, to one another. Any two of more of the diffraction gratings may be arranged at an angle that is within a range defined by any two of the preceding values.

For instance, the device 100 may comprise first, second, and third diffraction gratings applied to the surface of the device. The first diffraction grating may impart a red hue to the device. The second diffraction grating may impart a green hue to the device. The third diffraction grating may impart a blue hue to the device. The red, green, and blue hues may be chosen to impart a desired color to the device. The desired color may be chosen from a color chart, such as any color chart described herein. For instance, the desired color may be chosen from an International Commission on Illumination (CIE) color chart such as that described herein with respect to FIG. 1C or a condensed CIE color chart such as that described herein with respect to FIG. 1D. The desired color may be detected through the use of an optical spectrometer or a digital camera. The desired color may correspond to the color of an iris or pupil of one or more eyes of a wearer of the device.

In another example, the device 100 may comprise first, second, third, fourth, fifth, and sixth diffraction gratings applied to the surface of the device. The first and fourth diffraction gratings may form a cross grating pair. For instance, the first and fourth diffraction gratings may be substantially perpendicular to one another. The first and fourth diffraction grating may have optical parameters selected such that they impart the same or a similar color to the device, such as a red hue. Similarly, the second and fifth diffraction gratings may form a cross grating pair. For instance, the second and fifth diffraction gratings may be substantially perpendicular to one another. The second and fifth diffraction grating may have optical parameters selected such that they impart the same or a similar color to the device, such as a green hue. The third and sixth diffraction gratings may form a cross grating pair. For instance, the third and sixth diffraction gratings may be substantially perpendicular to one another. The third and sixth diffraction grating may have optical parameters selected such that they impart the same or a similar color to the device, such as a blue hue. The use of cross gratings may increase the efficiency of the optical effects produced by the gratings.

The diffraction grating 110 may be produced by any of the methods described herein, such as any of methods 200, 300, 400, 500, and 600 described herein. For instance, the diffraction grating may be imprinted on the surface of the device. The diffraction grating may comprise a plurality of regions that have been ablated from the surface of the device. The diffraction grating may comprise a lithographically patterned phase change material, such as a lithographically patterned photopolymer.

FIG. 1C shows a first color chart of colors that may be imparted to a wearable ocular device using the systems and methods described herein. The color chart may comprise a CIE color chart. The CIE color chart may be used to select a color to be imparted to a wearable ocular device described herein using any of the methods described herein.

FIG. 1D shows a second color chart of colors that may be imparted to a wearable ocular device using the systems and methods described herein. The color chart may comprise a condensed CIE color chart. The condensed CIE color chart may be used to select a color to be imparted to a wearable ocular device described herein using any of the methods described herein.

Wearable ocular devices of the present disclosure may have therapeutic applications. For instance, the device 100 may provide corneal protection for wearers that suffer conditions such as entropion, trichiasis, tarsal scarring, recurrent corneal erosion, or post-surgical ptosis. The device 100 may provide corneal pain relief for wearers that suffer conditions such as bullous keratopathy, epithelial erosion, epithelial abrasion, filamentary keratitis, or post-keratoplasty. The device 100 may be used as a bandage during a healing process for conditions such as chronic epithelial defect, corneal ulcer, neurotrophic keratitis, neuroparalytic keratitis, chemical burn, or post-surgical epithelial defect. The device 100 may be used as a bandage during a healing process after an ocular surgery such as small incision lenticule extraction (SMILE), laser-assisted in situ keratomileusis (LASIK), laser epithelial keratomileusis (LASEK), photorefractive keratectomy (PRK), penetrating keratoplasty (PK), phototherapeutic keratectomy (PTK), automated lamellar keratoplasty (ALK), refractive lens exchange (RLE), presbyopic lens exchange (PRELEX), lamellar graft, corneal flap, or other corneal surgical conditions. The device 100 may be used to provide optical correction during a healing procedure if such optical correction is necessary or desired.

The device 100 may be used to mask or camouflage a condition such as aniridia, pupil irregularity, permanent eye damage, or amblyopia in order to improve the appearance of a wearer of the device or to improve quality of life for a wearer of the device. The device 100 may be used to mitigate or eliminate double vision or to mitigate or eliminate the need for an occluder lens. In such an application, the device 100 may comprise a solid black pupillary component on an inner portion of the device (which may have a diameter of 1-4 mm larger that a maximum pupil size of an eye of a wearer of the device) to block out light and a clear outer edge on an outer portion of the device. The diameter of the solid black pupillary component may be selected based on measurements of the maximum size of the pupil obtained in dim light conditions. The device 100 may be used to mitigate or eliminate photophobia. In such an application, the device 100 may comprise a prosthetic iris lens in an inner portion of the device (thereby mitigating or eliminating light sensitivity) and a clear outer edge on an outer portion of the device. The prosthetic iris lens may have a diameter that is large enough to ensure coverage of the disfigured iris of a wearer of the device. The device 100 may be used to enhance contrast or vision. For instance, the device 100 may be used to create a sunglass effect whereby brightness of light received by an eye of a wearer of the device is reduced. The device 100 may be used to increase or maximize contrast by applying a color tint (such as a gray, green, or amber tint) to the device. Such contrast-enhanced devices may be particularly useful to athletes in enhancing their athletic performance. The device 100 may be used to correct color vision deficiencies, such as by providing a red tint to the device.

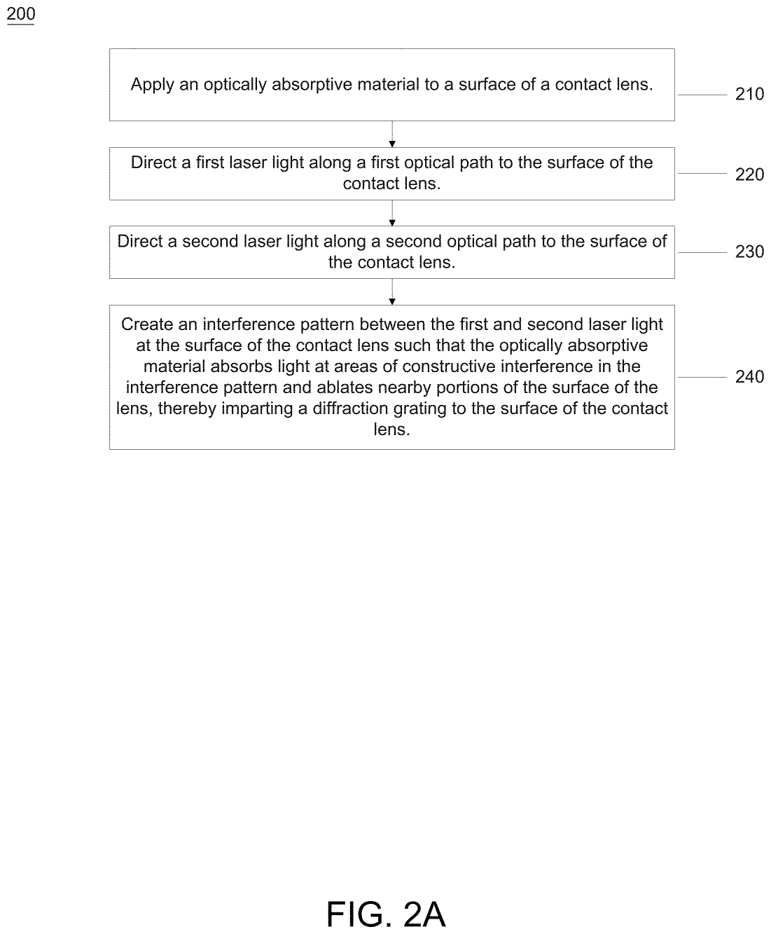

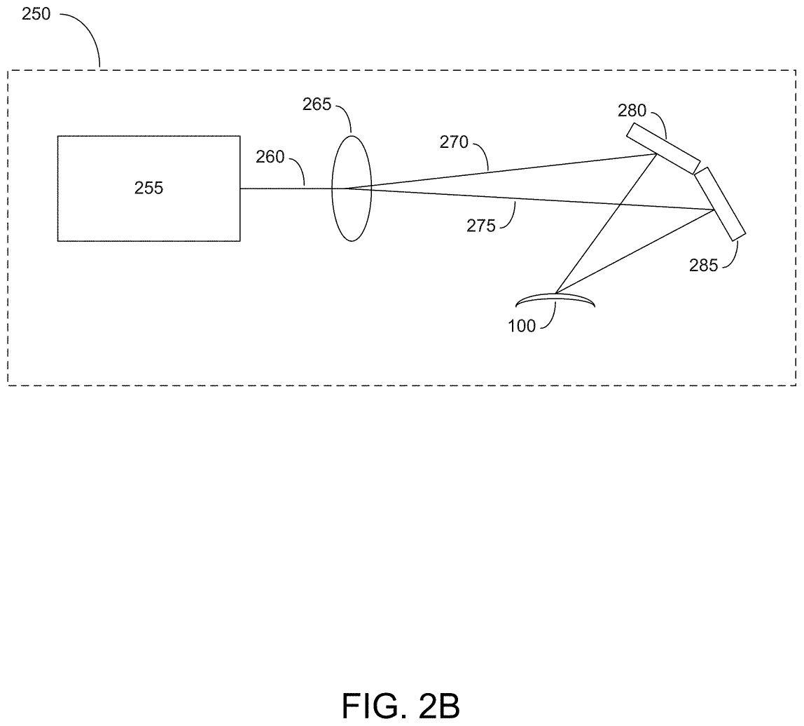

FIG. 2A shows a flowchart for a method 200 of imparting a representation to a wearable ocular device using transmission holography ablation to produce a diffraction grating on a surface of the device. In a first operation 210, the method 200 may comprise applying an optically absorptive material to a surface of the device. The device may be any device described herein. The device may be a contact lens. The surface of the device may be a front surface of the contact lens. The surface of the device may be a back surface of the contact lens. The device may be bifocals. The device may be an ocular prosthesis. The optically absorptive material may absorb light and heat, resulting in the removal of material from the surface of the device by ablation or sublimation. The optically absorptive material may comprise an ink. The optically absorptive material may comprise a dye. The optically absorptive material may be a thin film. The optically absorptive material may be a thin film. The optically absorptive material may have a thickness of at least 1 nanometer (nm), at least 2 nm, at least 3 nm, at least 4 nm, at least 5 nm, at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, at least 200 nm, at least 300 nm, at least 400 nm, at least 500 nm, at least 600 nm, at least 700 nm, at least 800 nm, at least 900 nm, at least 1 micrometer (μm), at least 2 μm, at least 3 μm, at least 4 μm, at least 5 μm, at least 6 μm, at least 7 μm, at least 8 μm, at least 9 μm, at least 10 μm, at least 20 μm, at least 30 μm, at least 40 μm, at least 50 μm, at least 60 μm, at least 70 μm, at least 80 μm, at least 90 μm, at least 100 μm, at least 200 μm, at least 300 μm, at least 400 μm, at least 500 μm, at least 600 μm, at least 700 μm, at least 800 μm, at least 900 μm, or at least 1,000 μm, or more. The optically absorptive material may have a thickness of at most 1,000 μm, at most 900 μm, at most 800 μm, at most 700 μm, at most 600 μm, at most 500 μm, at most 400 μm, at most 300 μm, at most 200 μm, at most 100 μm, at most 90 μm, at most 80 μm, at most 70 μm, at most 60 μm, at most 50 μm, at most 40 μm, at most 30 μm, at most 20 μm, at most 10 μm, at most 9 μm, at most 8 μm, at most 7 μm, at most 6 μm, at most 5 μm, at most 4 μm, at most 3 μm, at most 2 μm, at most 1 μm, at most 900 nm, at most 800 nm, at most 700 nm, at most 600 nm, at most 500 nm, at most 400 nm, at most 300 nm, at most 200 nm, at most 100 nm, at most 90 nm, at most 80 nm, at most 70 nm, at most 60 nm, at most 50 nm, at most 40 nm, at most 30 nm, at most 20 nm, at most 10 nm, at most 9 nm, at most 8 nm, at most 7 nm, at most 6 nm, at most 5 nm, at most 4 nm, at most 3 nm, at most 2 nm, at most 1 nm, or less. The optically absorptive material may have a thickness that is within a range defined by any two of the preceding values.

In a second operation 220, the method 200 may comprise directing a first laser light along a first optical path to the surface of the device. The first laser light may be emitted by a laser. The first laser light may be emitted by a continuous wave laser. The first laser light may be emitted by a pulsed laser. The first laser light may be emitted by a gas laser, such as a helium-neon (HeNe) laser, an argon (Ar) laser, a krypton (Kr) laser, a xenon (Xe) ion laser, a nitrogen (N₂) laser, a carbon dioxide (CO₂) laser, a carbon monoxide (CO) laser, a transversely excited atmospheric (TEA) laser, or an excimer laser. For instance, the first laser light may be emitted by an argon dimer (Ar₂) excimer laser, a krypton dimer (Kr₂) excimer laser, a fluorine dimer (F₂) excimer laser, a xenon dimer (Xe₂) excimer laser, an argon fluoride (ArF) excimer laser, a krypton chloride (KrCl) excimer laser, a krypton fluoride (KrF) excimer laser, a xenon bromide (XeBr) excimer laser, a xenon chloride (XeCl) excimer laser, or a xenon fluoride (XeF) excimer laser. The first laser light may be emitted by a dye laser.

The first laser light may be emitted by a metal-vapor laser, such as a helium-cadmium (HeCd) metal-vapor laser, a helium-mercury (HeHg) metal-vapor laser, a helium-selenium (HeSe) metal-vapor laser, a helium-silver (HeAg) metal-vapor laser, a strontium (Sr) metal-vapor laser, a neon-copper (NeCu) metal-vapor laser, a copper (Cu) metal-vapor laser, a gold (Au) metal-vapor laser, a manganese (Mn) metal-vapor, or a manganese chloride (MnCl₂) metal-vapor laser.

The first laser light may be emitted by a solid-state laser, such as a ruby laser, a metal-doped crystal laser, or a metal-doped fiber laser. For instance, the first laser light may be emitted by a neodymium-doped yttrium aluminum garnet (Nd:YAG) laser, a neodymium/chromium doped yttrium aluminum garnet (Nd/Cr:YAG) laser, an erbium-doped yttrium aluminum garnet (Er:YAG) laser, a neodymium-doped yttrium lithium fluoride (Nd:YLF) laser, a neodymium-doped yttrium orthovanadate (ND:YVO₄) laser, a neodymium-doped yttrium calcium oxoborate (Nd:YCOB) laser, a neodymium glass (Nd:glass) laser, a titanium sapphire (Ti:sapphire) laser, a thulium-doped ytrrium aluminum garnet (Tm:YAG) laser, a ytterbium-doped ytrrium aluminum garnet (Yb:YAG) laser, a ytterbium-doped glass (Yt:glass) laser, a holmium ytrrium aluminum garnet (Ho:YAG) laser, a chromium-doped zinc selenide (Cr:ZnSe) laser, a cerium-doped lithium strontium aluminum fluoride (Ce:LiSAF) laser, a cerium-doped lithium calcium aluminum fluoride (Ce:LiCAF) laser, a erbium-doped glass (Er:glass), an erbium-ytterbium-codoped glass (Er/Yt:glass) laser, a uranium-doped calcium fluoride (U:CaF₂) laser, or a samarium-doped calcium fluoride (Sm:CaF₂) laser.

The first laser light may be emitted by a semiconductor laser or diode laser, such as a gallium nitride (GaN) laser, an indium gallium nitride (InGaN) laser, an aluminum gallium indium phosphide (AlGaInP) laser, an aluminum gallium arsenide (AlGaAs) laser, an indium gallium arsenic phosphide (InGaAsP) laser, a vertical cavity surface emitting laser (VCSEL), or a quantum cascade laser.

The first laser light may be continuous wave laser light. The first laser light may be pulsed laser light. The first laser light may have a pulse length of at least 1 femtoseconds (fs), at least 2 fs, at least 3 fs, at least 4 fs, at least 5 fs, at least 6 fs, at least 7 fs, at least 8 fs, at least 9 fs, at least 10 fs, at least 20 fs, at least 30 fs, at least 40 fs, at least 50 fs, at least 60 fs, at least 70 fs, at least 80 fs, at least 90 fs, at least 100 fs, at least 200 fs, at least 300 fs, at least 400 fs, at least 500 fs, at least 600 fs, at least 700 fs, at least 800 fs, at least 900 fs, at least 1 picosecond (ps), at least 2 ps, at least 3 ps, at least 4 ps, at least 5 ps, at least 6 ps, at least 7 ps, at least 8 ps, at least 9 ps, at least 10 ps, at least 20 ps, at least 30 ps, at least 40 ps, at least 50 ps, at least 60 ps, at least 70 ps, at least 80 ps, at least 90 ps, at least 100 ps, at least 200 ps, at least 300 ps, at least 400 ps, at least 500 ps, at least 600 ps, at least 700 ps, at least 800 ps, at least 900 ps, at least 1 nanosecond (ns), at least 2 ns, at least 3 ns, at least 4 ns, at least 5 ns, at least 6 ns, at least 7 ns, at least 8 ns, at least 9 ns, at least 10 ns, at least 20 ns, at least 30 ns, at least 40 ns, at least 50 ns, at least 60 ns, at least 70 ns, at least 80 ns, at least 90 ns, at least 100 ns, at least 200 ns, at least 300 ns, at least 400 ns, at least 500 ns, at least 600 ns, at least 700 ns, at least 800 ns, at least 900 ns, at least 1,000 ns, or more. The first laser light may have a pulse length of at most 1,000 ns, at most 900 ns, at most 800 ns, at most 700 ns, at most 600 ns, at most 500 ns, at most 400 ns, at most 300 ns, at most 200 ns, at most 100 ns, at most 90 ns, at most 80 ns, at most 70 ns, at most 60 ns, at most 50 ns, at most 40 ns, at most 30 ns, at most 20 ns, at most 10 ns, at most 9 ns, at most 8 ns, at most 7 ns, at most 6 ns, at most 5 ns, at most 4 ns, at most 3 ns, at most 2 ns, at most 1 ns, at most 900 ps, at most 800 ps, at most 700 ps, at most 600 ps, at most 500 ps, at most 400 ps, at most 300 ps, at most 200 ps, at most 100 ps, at most 90 ps, at most 80 ps, at most 70 ps, at most 60 ps, at most 50 ps, at most 40 ps, at most 30 ps, at most 20 ps, at most 10 ps, at most 9 ps, at most 8 ps, at most 7 ps, at most 6 ps, at most 5 ps, at most 4 ps, at most 3 ps, at most 2 ps, at most 1 ps, at most 900 fs, at most 800 fs, at most 700 fs, at most 600 fs, at most 500 fs, at most 400 fs, at most 300 fs, at most 200 fs, at most 100 fs, at most 90 fs, at most 80 fs, at most 70 fs, at most 60 fs, at most 50 fs, at most 40 fs, at most 30 fs, at most 20 fs, at most 10 fs, at most 9 fs, at most 8 fs, at most 7 fs, at most 6 fs, at most 5 fs, at most 4 fs, at most 3 fs, at most 2 fs, at most 1 fs, or less. The first laser light may have a pulse length that is within a range defined by any two of the preceding values. For instance, the first laser light may have a pulse length between 1 ns and 50 ns.

The first laser light may have a repetition rate of at least 1 hertz (Hz), at least 2 Hz, at least 3 Hz, at least 4 Hz, at least 5 Hz, at least 6 Hz, at least 7 Hz, at least 8 Hz, at least 9 Hz, at least 10 Hz, at least 20 Hz, at least 30 Hz, at least 40 Hz, at least 50 Hz, at least 60 Hz, at least 70 Hz, at least 80 Hz, at least 90 Hz, at least 100 Hz, at least 200 Hz, at least 300 Hz, at least 400 Hz, at least 500 Hz, at least 600 Hz, at least 700 Hz, at least 800 Hz, at least 900 Hz, at least 1 kilohertz (kHz), at least 2 kHz, at least 3 kHz, at least 4 kHz, at least 5 kHz, at least 6 kHz, at least 7 kHz, at least 8 kHz, at least 9 kHz, at least 10 kHz, at least 20 kHz, at least 30 kHz, at least 40 kHz, at least 50 kHz, at least 60 kHz, at least 70 kHz, at least 80 kHz, at least 90 kHz, at least 100 kHz, at least 200 kHz, at least 300 kHz, at least 400 kHz, at least 500 kHz, at least 600 kHz, at least 700 kHz, at least 800 kHz, at least 900 kHz, at least 1 megahertz (MHz), at least 2 MHz, at least 3 MHz, at least 4 MHz, at least 5 MHz, at least 6 MHz, at least 7 MHz, at least 8 MHz, at least 9 MHz, at least 10 MHz, at least 20 MHz, at least 30 MHz, at least 40 MHz, at least 50 MHz, at least 60 MHz, at least 70 MHz, at least 80 MHz, at least 90 MHz, at least 100 MHz, at least 200 MHz, at least 300 MHz, at least 400 MHz, at least 500 MHz, at least 600 MHz, at least 700 MHz, at least 800 MHz, at least 900 MHz, at least 1,000 MHz, or more. The first laser light may have a repetition rate of at most 1,000 MHz, at most 900 MHz, at most 800 MHz, at most 700 MHz, at most 600 MHz, at most 500 MHz, at most 400 MHz, at most 300 MHz, at most 200 MHz, at most 100 MHz, at most 90 MHz, at most 80 MHz, at most 70 MHz, at most 60 MHz, at most 50 MHz, at most 40 MHz, at most 30 MHz, at most 20 MHz, at most 10 MHz, at most 9 MHz, at most 8 MHz, at most 7 MHz, at most 6 MHz, at most 5 MHz, at most 4 MHz, at most 3 MHz, at most 2 MHz, at most 1 MHz, at most 900 kHz, at most 800 kHz, at most 700 kHz, at most 600 kHz, at most 500 kHz, at most 400 kHz, at most 300 kHz, at most 200 kHz, at most 100 kHz, at most 90 kHz, at most 80 kHz, at most 70 kHz, at most 60 kHz, at most 50 kHz, at most 40 kHz, at most 30 kHz, at most 20 kHz, at most 10 kHz, at most 9 kHz, at most 8 kHz, at most 7 kHz, at most 6 kHz, at most 5 kHz, at most 4 kHz, at most 3 kHz, at most 2 kHz, at most 1 kHz, at most 900 Hz, at most 800 Hz, at most 700 Hz, at most 600 Hz, at most 500 Hz, at most 400 Hz, at most 300 Hz, at most 200 Hz, at most 100 Hz, at most 90 Hz, at most 80 Hz, at most 70 Hz, at most 60 Hz, at most 50 Hz, at most 40 Hz, at most 30 Hz, at most 20 Hz, at most 10 Hz, at most 9 Hz, at most 8 Hz, at most 7 Hz, at most 6 Hz, at most 5 Hz, at most 4 Hz, at most 3 Hz, at most 2 Hz, at most 1 Hz, or less. The first laser light may have a repetition rate that is within a range defined by any two of the preceding values.

The first laser light may have a pulse energy of at least 1 nanojoule (nJ), at least 2 nJ, at least 3 nJ, at least 4 nJ, at least 5 nJ, at least 6 nJ, at least 7 nJ, at least 8 nJ, at least 9 nJ, at least 10 nJ, at least 20 nJ, at least 30 nJ, at least 40 nJ, at least 50 nJ, at least 60 nJ, at least 70 nJ, at least 80 nJ, at least 90 nJ, at least 100 nJ, at least 200 nJ, at least 300 nJ, at least 400 nJ, at least 500 nJ, at least 600 nJ, at least 700 nJ, at least 800 nJ, at least 900 nJ, at least 1 microjoule (μJ), at least 2 μJ, at least 3 μJ, at least 4 μJ, at least 5 μJ, at least 6 μJ, at least 7 μJ, at least 8 μJ, at least 9 μJ, at least 10 μJ, at least 20 μJ, at least 30 μJ, at least 40 μJ, at least 50 μJ, at least 60 μJ, at least 70 μJ, at least 80 μJ, at least 90 μJ, at least 100 μJ, at least 200 μJ, at least 300 μJ, at least 400 μJ, at least 500 μJ, at least 600 μJ, at least 700 μJ, at least 800 μJ, at least 900 μJ, a least 1 millijoule (mJ), at least 2 mJ, at least 3 mJ, at least 4 mJ, at least 5 mJ, at least 6 mJ, at least 7 mJ, at least 8 mJ, at least 9 mJ, at least 10 mJ, at least 20 mJ, at least 30 mJ, at least 40 mJ, at least 50 mJ, at least 60 mJ, at least 70 mJ, at least 80 mJ, at least 90 mJ, at least 100 mJ, at least 200 mJ, at least 300 mJ, at least 400 mJ, at least 500 mJ, at least 600 mJ, at least 700 mJ, at least 800 mJ, at least 900 mJ, a least 1 Joule (J), or more. The first laser light may have a pulse energy of at most 1 J, at most 900 mJ, at most 800 mJ, at most 700 mJ, at most 600 mJ, at most 500 mJ, at most 400 mJ, at most 300 mJ, at most 200 mJ, at most 100 mJ, at most 90 mJ, at most 80 mJ, at most 70 mJ, at most 60 mJ, at most 50 mJ, at most 40 mJ, at most 30 mJ, at most 20 mJ, at most 10 mJ, at most 9 mJ, at most 8 mJ, at most 7 mJ, at most 6 mJ, at most 5 mJ, at most 4 mJ, at most 3 mJ, at most 2 mJ, at most 1 mJ, at most 900 μJ, at most 800 μJ, at most 700 μJ, at most 600 μJ, at most 500 μJ, at most 400 μJ, at most 300 μJ, at most 200 μJ, at most 100 μJ, at most 90 μJ, at most 80 μJ, at most 70 μJ, at most 60 μJ, at most 50 μJ, at most 40 μJ, at most 30 μJ, at most 20 μJ, at most 10 μJ, at most 9 μJ, at most 8 μJ, at most 7 μJ, at most 6 μJ, at most 5 μJ, at most 4μJ, at most 3 μJ, at most 2 μJ, at most 1 μJ, at most 900 nJ, at most 800 nJ, at most 700 nJ, at most 600 nJ, at most 500 nJ, at most 400 nJ, at most 300 nJ, at most 200 nJ, at most 100 nJ, at most 90 nJ, at most 80 nJ, at most 70 nJ, at most 60 nJ, at most 50 nJ, at most 40 nJ, at most 30 nJ, at most 20 nJ, at most 10 nJ, at most 9 nJ, at most 8 nJ, at most 7 nJ, at most 6 nJ, at most 5 nJ, at most 4 nJ, at most 3 nJ, at most 2 nJ, at most 1 nJ, or less. The first laser light may have a pulse energy that is within a range defined by any two of the preceding values. For instance, the first laser light may have a pulse energy between 100 mJ and 500 mJ.

The first laser light may have an average power of at least 1 microwatt (μW), at least 2 μW, at least 3 μW, at least 4 μW, at least 5 μW, at least 6 μW, at least 7 μW, at least 8 μW, at least 9 μW, at least 10 μW, at least 20 μW, at least 30 μW, at least 40 μW, at least 50 μW, at least 60 μW, at least 70 μW, at least 80 μW, at least 90 μW, at least 100 μW, at least 200 μW, at least 300 μW, at least 400 μW, at least 500 μW, at least 600 μW, at least 700 μW, at least 800 μW, at least 900 μW, at least 1 milliwatt (mW), at least 2 mW, at least 3 mW, at least 4 mW, at least 5 mW, at least 6 mW, at least 7 mW, at least 8 mW, at least 9 mW, at least 10 mW, at least 20 mW, at least 30 mW, at least 40 mW, at least 50 mW, at least 60 mW, at least 70 mW, at least 80 mW, at least 90 mW, at least 100 mW, at least 200 mW, at least 300 mW, at least 400 mW, at least 500 mW, at least 600 mW, at least 700 mW, at least 800 mW, at least 900 mW, at least 1 watt (W), at least 2 W, at least 3 W, at least 4 W, at least 5 W, at least 6 W, at least 7 W, at least 8 W, at least 9 W, at least 10 W, at least 20 W, at least 30 W, at least 40 W, at least 50 W, at least 60 W, at least 70 W, at least 80 W, at least 90 W, at least 100 W, at least 200 W, at least 300 W, at least 400 W, at least 500 W, at least 600 W, at least 700 W, at least 800W, at least 900 W, at least 1,000 W, or more. The first laser light may have an average power of at most 1,000 W, at most 900 W, at most 800 W, at most 700 W, at most 600 W, at most 500 W, at most 400 W, at most 300 W, at most 200 W, at most 100 W, at most 90 W, at most 80 W, at most 70 W, at most 60 W, at most 50 W, at most 40 W, at most 30 W, at most 20 W, at most 10 W, at most 9 W, at most 8 W, at most 7 W, at most 6 W, at most 5 W, at most 4 W, at most 3 W, at most 2 W, at most 1 W, at most 900 mW, at most 800 mW, at most 700 mW, at most 600 mW, at most 500 mW, at most 400 mW, at most 300 mW, at most 200 mW, at most 100 mW, at most 90 mW, at most 80 mW, at most 70 mW, at most 60 mW, at most 50 mW, at most 40 mW, at most 30 mW, at most 20 mW, at most 10 mW, at most 9 mW, at most 8 mW, at most 7 mW, at most 6 mW, at most 5 mW, at most 4 mW, at most 3 mW, at most 2 mW, at most 1 mW, at most 900 μW, at most 800 μW, at most 700 μW, at most 600 μW, at most 500 μW, at most 400 μW, at most 300 μW, at most 200 μW, at most 100 μW, at most 90 μW, at most 80 μW, at most 70 μW, at most 60 μW, at most 50 μW, at most 40 μW, at most 30 μW, at most 20 μW, at most 10 μW, at most 9 μW, at most 8 μW, at most 7 μW, at most 6 μW, at most 5 μW, at most 4 μW, at most 3 μW, at most 2 μW, at most 1 μW, or more. The first laser light may have a power that is within a range defined by any two of the preceding values.

The first laser light may comprise a wavelength in the ultraviolet (UV), visible, or infrared (IR) portions of the electromagnetic spectrum. The first laser light may comprise a wavelength of at least 100 nanometers (nm), at least 110 nm, at least 120 nm, at least 130 nm, at least 140 nm, at least 150 nm, at least 160 nm, at least 170 nm, at least 180 nm, at least 190 nm, at least 200 nm, at least 210 nm, at least 220 nm, at least 230 nm, at least 240 nm, at least 250 nm, at least 260 nm, at least 270 nm, at least 280 nm, at least 290 nm, at least 300 nm, at least 310 nm, at least 320 nm, at least 330 nm, at least 340 nm, at least 350 nm, at least 360 nm, at least 370 nm, at least 380 nm, at least 390 nm, at least 400 nm, at least 410 nm, at least 420 nm, at least 430 nm, at least 440 nm, at least 450 nm, at least 460 nm, at least 470 nm, at least 480 nm, at least 490 nm, at least 500 nm, at least 510 nm, at least 520 nm, at least 530 nm, at least 540 nm, at least 550 nm, at least 560 nm, at least 570 nm, at least 580 nm, at least 590 nm, at least 600 nm, at least 610 nm, at least 620 nm, at least 630 nm, at least 640 nm, at least 650 nm, at least 660 nm, at least 670 nm, at least 680 nm, at least 690 nm, at least 700 nm, at least 710 nm, at least 720 nm, at least 730 nm, at least 740 nm, at least 750 nm, at least 760 nm, at least 770 nm, at least 780 nm, at least 790 nm, at least 800 nm, at least 810 nm, at least 820 nm, at least 830 nm, at least 840 nm, at least 850 nm, at least 860 nm, at least 870 nm, at least 880 nm, at least 890 nm, at least 900 nm, at least 910 nm, at least 920 nm, at least 930 nm, at least 940 nm, at least 950 nm, at least 960 nm, at least 970 nm, at least 980 nm, at least 990 nm, at least 1,000 nm, at least 1,010 nm, at least 1,020 nm, at least 1,030 nm, at least 1,040 nm, at least 1,050 nm, at least 1,060 nm, at least 1,070 nm, at least 1,080 nm, at least 1,090 nm, at least 1,100 nm, at least 1,110 nm, at least 1,120 nm, at least 1,130 nm, at least 1,140 nm, at least 1,150 nm, at least 1,160 nm, at least 1,170 nm, at least 1,180 nm, at least 1,190 nm, at least 1,200 nm, at least 1,210 nm, at least 1,220 nm, at least 1,230 nm, at least 1,240 nm, at least 1,250 nm, at least 1,260 nm, at least 1,270 nm, at least 1,280 nm, at least 1,290 nm, at least 1,300 nm, at least 1,310 nm, at least 1,320 nm, at least 1,330 nm, at least 1,340 nm, at least 1,350 nm, at least 1,360 nm, at least 1,370 nm, at least 1,380 nm, at least 1,390 nm, at least 1,400 nm, or more. The first laser light may comprise a wavelength of at most 1,400 nm, at most 1,390 nm, at most 1,380 nm, at most 1,370 n, at most 1,360 nm, at most 1,350 nm, at most 1,340 nm, at most 1,330 nm, at most 1,320 nm, at most 1,310 nm, at most 1,300 nm, at most 1,290 nm, at most 1,280 nm, at most 1,270 n, at most 1,260 nm, at most 1,250 nm, at most 1,240 nm, at most 1,230 nm, at most 1,220 nm, at most 1,210 nm, at most 1,200 nm, at most 1,190 nm, at most 1,180 nm, at most 1,170 n, at most 1,160 nm, at most 1,150 nm, at most 1,140 nm, at most 1,130 nm, at most 1,120 nm, at most 1,110 nm, at most 1,100 nm, at most 1,090 nm, at most 1,080 nm, at most 1,070 n, at most 1,060 nm, at most 1,050 nm, at most 1,040 nm, at most 1,030 nm, at most 1,020 nm, at most 1,010 nm, at most 1,000 nm, at most 990 nm, at most 980 nm, at most 970 nm, at most 960 nm, at most 950 nm, at most 940 nm, at most 930 nm, at most 920 nm, at most 910 nm, at most 900 nm, at most 890 nm, at most 880 nm, at most 870 nm, at most 860 nm, at most 850 nm, at most 840 nm, at most 830 nm, at most 820 nm, at most 810 nm, at most 800 nm, at most 790 nm, at most 780 nm, at most 770 nm, at most 760 nm, at most 750 nm, at most 740 nm, at most 730 nm, at most 720 nm, at most 710 nm, at most 700 nm, at most 690 nm, at most 680 nm, at most 670 nm, at most 660 nm, at most 650 nm, at most 640 nm, at most 630 nm, at most 620 nm, at most 610 nm, at most 600 nm, at most 590 nm, at most 580 nm, at most 570 nm, at most 560 nm, at most 550 nm, at most 540 nm, at most 530 nm, at most 520 nm, at most 510 nm, at most 500 nm, at most 490 nm, at most 480 nm, at most 470 nm, at most 460 nm, at most 450 nm, at most 440 nm, at most 430 nm, at most 420 nm, at most 410 nm, at most 400 nm, at most 390 nm, at most 380 nm, at most 370 nm, at most 360 nm, at most 350 nm, at most 340 nm, at most 330 nm, at most 320 nm, at most 310 nm, at most 300 nm, at most 290 nm, at most 280 nm, at most 270 nm, at most 260 nm, at most 250 nm, at most 240 nm, at most 230 nm, at most 220 nm, at most 210 nm, at most 200 nm, at most 190 nm, at most 180 nm, at most 170 nm, at most 160 nm, at most 150 nm, at most 140 nm, at most 130 nm, at most 120 nm, at most 110 nm, at most 100 nm, or less. The first laser light may comprise a wavelength that is within a range defined by any two of the preceding values.

The first laser light may have a bandwidth of at least 0.001 nm, at least 0.002 nm, at least 0.003 nm, at least 0.004 nm, at least 0.005 nm, at least 0.006 nm, at least 0.007 nm, at least 0.008 nm, at least 0.009 nm, at least 0.01 nm, at least 0.02 nm, at least 0.03 nm, at least 0.04 nm, at least 0.05 nm, at least 0.06 nm, at least 0.07 nm, at least 0.08 nm, at least 0.09 nm, at least 0.1 nm, at least 0.2 nm, at least 0.3 nm, at least 0.4 nm, at least 0.5 nm, at least 0.6 nm, at least 0.7 nm, at least 0.8 nm, at least 0.9 nm, at least 1 nm, at least 2 nm, at least 3 nm, at least 4 nm, at least 5 nm, at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, or more. The first laser light may have a bandwidth of at most 100 nm, at most 90 nm, at most 80 nm, at most 70 nm, at most 60 nm, at most 50 nm, at most 40 nm, at most 30 nm, at most 20 nm, at most 10 nm, at most 9 nm, at most 8 nm, at most 7 nm, at most 6 nm, at most 5 nm, at most 4 nm, at most 3 nm, at most 2 nm, at most 1 nm, at most 0.9 nm, at most 0.8 nm, at most 0.7 nm, at most 0.6 nm, at most 0.5 nm, at most 0.4 nm, at most 0.3 nm, at most 0.2 nm, at most 0.1 nm, at most 0.09 nm, at most 0.08 nm, at most 0.07 nm, at most 0.06 nm, at most 0.05 nm, at most 0.04 nm, at most 0.03 nm, at most 0.02 nm, at most 0.01 nm, at most 0.009 nm, at most 0.008 nm, at most 0.007 nm, at most 0.006 nm, at most 0.005 nm, at most 0.004 nm, at most 0.003 nm, at most 0.002 nm, at most 0.001 nm, or less. The first laser light may have a bandwidth that is within a range defined by any two of the preceding values.

The first laser light may have a diameter (for instance, as measured by a Rayleigh beam width, full width at half maximum, 1/e² width, second moment width, knife-edge width, D86 width, or any other measure of beam diameter) of at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1 mm, at least 2 mm, at least 3 mm, at least 4 mm, at least 5 mm, at least 6 mm, at least 7 mm, at least 8 mm, at least 9 mm, at least 10 mm, at least 20 mm, at least 30 mm, at least 40 mm, at least 50 mm, at least 60 mm, at least 70 mm, at least 80 mm, at least 90 mm, at least 100 mm, or more. The first light may have a diameter of at most 100 mm, at most 90 mm, at most 80 mm, at most 70 mm, at most 60 mm, at most 50 mm, at most 40 mm, at most 30 mm, at most 20 mm, at most 10 mm, at most 9 mm, at most 8 mm, at most 7 mm, at most 6 mm, at most 5 mm, at most 4 mm, at most 3 mm, at most 2 mm, at most 1 mm, at most 0.9 mm, at most 0.8 mm, at most 0.7 mm, at most 0.6 mm, at most 0.5 mm, at most 0.4 mm, at most 0.3 mm, at most 0.2 mm, at most 0.1 mm, or less. The first laser light may have a diameter that is within a range defined by any two of the preceding values. In some cases, the first laser light may have a diameter that is smaller than the diameter of a wearable ocular device. In some instances, the first laser light may have a diameter that is approximately equal to the diameter of a wearable ocular device. In still further instances, the first laser light may have a diameter that is larger than the diameter of a wearable ocular device. For instance, the first laser light may have a diameter that allows the first laser light to simultaneously illuminate a plurality of wearable ocular devices. Such a system may allow the simultaneous production of diffraction gratings on a plurality of wearable ocular devices in a batch process.

In a third operation 230, the method 200 may comprise directing a second laser light along a second optical path to the surface of the device. The second laser light may be similar to any first laser light described herein. The first and second laser light may be emitted by different lasers. The first and second laser light may be emitted by the same laser.

The first and second laser light may be directed along the first and second optical paths, respectively, by a spatial filter. The spatial filter may comprise a lens.

The first optical path may comprise a reference mirror. The spatial filter and first optical path may be configured such that the first laser light is directed from the spatial filter to the reference mirror. The first laser light may be directed from the reference mirror to a first portion of the surface of the device.

The second optical path may comprise an objective mirror. The spatial filter and second optical path may be configured such that the second laser light is directed from the spatial filter to the objective mirror. The second laser light may be directed from the objective mirror to a second portion of the surface of the device. The first and second portions of the surface of the device may be distinct. The first and second portions of the surface of the device may partially overlap. For instance, the first and second portions of the surface of the device may have first and second lateral areas, respectively, that overlap by at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or more. The first and second portions of the surface of the device may have first and second lateral areas, respectively, that overlap by at most 99%, at most 98%, at most 97%, at most 96%, at most 95%, at most 94%, at most 93%, at most 92%, at most 91%, at most 90%, at most 80%, at most 70%, at most 60%, at most 50%, at most 40%, at most 30%, at most 20%, at most 10%, at most 9%, at most 8%, at most 7%, at most 6%, at most 5%, at most 4%, at most 3%, at most 2%, at most 1%, or less. The first and second portions of the surface of the device may have first and second lateral areas, respectively, that overlap by a value that is within a range defined by any two of the preceding values. The first and second portions of the surface of the device may completely overlap.

In a fourth operation 240, the method 200 may comprise creating an interference pattern between the first and second laser light at the surface of the device such that the optically absorptive material absorbs light at areas of constructive interference in the interference pattern and ablates nearby portions of the surface of the device, thereby imparting a diffraction grating to the surface of the device.

The method 200 may be used to impart any representation described herein (such as any representation described herein with respect to FIG. 1A, 1B, 1C, or 1D) to the device. The representation may be an expression or designation.

The expression or designation may be a geometric object. For instance, the diffraction grating may cause a viewer of the device to perceive one or more dots, lines, shapes (such as one or more triangles, quadrilaterals, rectangles, squares, pentagons, hexagons, heptagons, octagons, nonagons, decagons, undecagons, dodecagons, polygons with more than 12 sides, ellipses, ovals, circles, or any other geometric shape). Such markings may represent an indication of one or more optically relevant parameters of the device, such as whether the device is properly centered or oriented on a wearer of the device. In some cases, the markings may represent an indication of whether a contact lens is properly centered or oriented on an eye of a wearer of a contact lens. For instance, the marking may comprise a bump or lenticular that indicates an orientation of the contact lens.

The expression or designation may be a repository of information. For instance, the diffraction grating may cause a viewer of the device to perceiver a barcode, a QR code, or a QR code with a circular hole in its center. The repository of information may be useful for quality control or other tracking purposes. For instance, the repository of information may enable tracking of the device during manufacturing or during an ophthalmological study or clinical trial.

The expression or designation may be a character or term. The character or term may be a character or term selected from any language, such as Mandarin, Spanish, English, Hindi, Arabic, Portuguese, Bengali, Russian, Japanese, Punjabi, German, Javanese, Wu, Malay, Telugu, Vietnamese, Korean, French, Marathi, Tamil, Urdu, Turkish, Italian, Yue, Cantonese, Thai, Gujarati, Jin, Min, Persian, Polish, Pashto, Kannada, Xiang, Malayalam, Sundanese, Hausa, Odia, Burmese, Hakka, Ukrainian, Bhojpuri, Tagalog, Yoruba, Maithili, Uzbek, Sindhi, Amharic, Fula, Romanian, Oromo, Igbo, Azerbaijani, Awadhi, Gan, Cebuano, Dutch, Kurdish, Serbo-Croatian, Malagasy, Saraiki, Nepali, Sinhalese, Chittagonian, Zhuang, Khmer, Turkmen, Assamese, Madurese, Somali, Marwari, Magahi, Haryanvi, Hungarian, Chhattisgarhi, Greek, Chewa, Deccan, Akan, Kazakh, Sylheti, Zulu, Czech, Kinyarwanda, Dhundhari, Haitian, Creole, Ilocano, Quechua, Kirundi, Swedish, Hmong, Shona, Uyghur, Hiligaynon, Ilonggo, Mossi, Xhosa, Belarusian, Balochi, Konkani, or any other language.

The expression or designation may be an image, such as one or more logos, brands, photographs, works of art, cartoons, or other images. The image may be obtained through an image scanning procedure.

The expression or designation may be configured to alter an appearance of a wearer of the device for artistic purposes, such as for use in movies or other live action performances. In some cases, the expression or designation may be configured to alter an appearance of an eye of a wearer of a contact lens for artistic purposes. For instance, the expression or designation may alter the appearance of the wearer's eye such that the wearer appears to have the eyes of an animal, monster, or other non-human.

The expression or designation may be a color.

The method 200 may further comprise repeating any 1, 2, 3, or 4 of operations 210, 220, 230, and 240 to impart a plurality of diffraction gratings to the surface of the device. The method 200 may further comprise repeating any 1, 2, 3, or 4 of operations 210, 220, 230, and 240 at least 1 time, at least 2 times, at least 3 times, at least 4 times, at least 5 times, at least 6 times, at least 7 times, at least 8 times, at least 9 times, at least 10 times, or more, to impart at least 1, at least 2, at least 3, at least 4, at least 5, at least 6, at least 7, at least 8, at least 9, at least 10, or more diffraction gratings to the surface of the device. The method 200 may further comprise repeating any 1, 2, 3, or 4 of operations 210, 220, 230, and 240 at most 10 times, at most 9 times, at most 8 times, at most 7 times, at most 6 times, at most 5 times, at most 4 times, at most 3 times, at most 2 times, or less, to impart at most 10, at most 9, at most 8, at most 7, at most 6, at most 5, at most 4, at most 3, at most 2, or fewer diffraction gratings to the surface of the device. The method 200 may further comprise repeating any 1, 2, 3, or 4 of operations 210, 220, 230, and 240 a number of times that is within a range defined by any two of the preceding values to impart a number of diffraction gratings that is within a range defined by any two of the preceding values to the surface of the device.

For instance, the method 200 may further comprise repeating any 1, 2, 3, or 4 of operations 210, 220, 230, and 240 a total of three times to impart first, second, and third diffraction gratings to the surface of the device. The first diffraction grating may impart a red hue to the device. The second diffraction grating may impart a green hue to the device. The third diffraction grating may impart a blue hue to the device. The red, green, and blue hues may be chosen to impart a desired color to the device. The desired color may be chosen from a color chart, such as any color chart described herein. For instance, the desired color may be chosen from a CIE color chart such as that described herein with respect to FIG. 1C or a condensed CIE color chart such as that described herein with respect to FIG. 1D.

The method 200 may further comprise, prior to operation 210, (i) selecting a desired color to be imparted to the device, and (ii) determining optical parameters required to produce the first, second, and third diffraction gratings.

The method 200 may further comprise, prior to operation 210, (i) using an optical spectrometer or digital camera to determine a desired color to be imparted to the device, and (ii) determining optical parameters required to produce the first, second, and third diffraction gratings.

The expression or designation may be an artificial pupil. The artificial pupil may comprise a moth eye structure.

The method 200 may further comprise removing the optically absorptive material from the surface of the device.

FIG. 2B shows an optical setup 250 for transmission holography ablation of a wearable ocular device. The optical setup 250 may be used to enact the method 200. The optical setup 250 may comprise a laser 255. The laser may be similar to any laser described herein. For instance, the laser may be similar to any laser described herein with respect to FIG. 2A. The laser may emit laser light 260. The laser light 260 may be similar to any laser light described herein. For instance, the laser light 260 may be similar to any first laser light described herein with respect to FIG. 2A. The laser light may be directed to an optical filter 265. The optical filter may comprise a lens. The optical filter may direct first laser light 270 along a first optical path to a reference mirror 280. The first laser light may be similar to any first laser light described herein, such as any first laser light described herein with respect to FIG. 2A. The optical filter may direct second laser light 275 along a second optical path to an objective mirror 285. The second laser light may be similar to any second laser light described herein, such as any second laser light described herein with respect to FIG. 2A. The first and second laser light may be directed to a surface of a wearable ocular device 100.

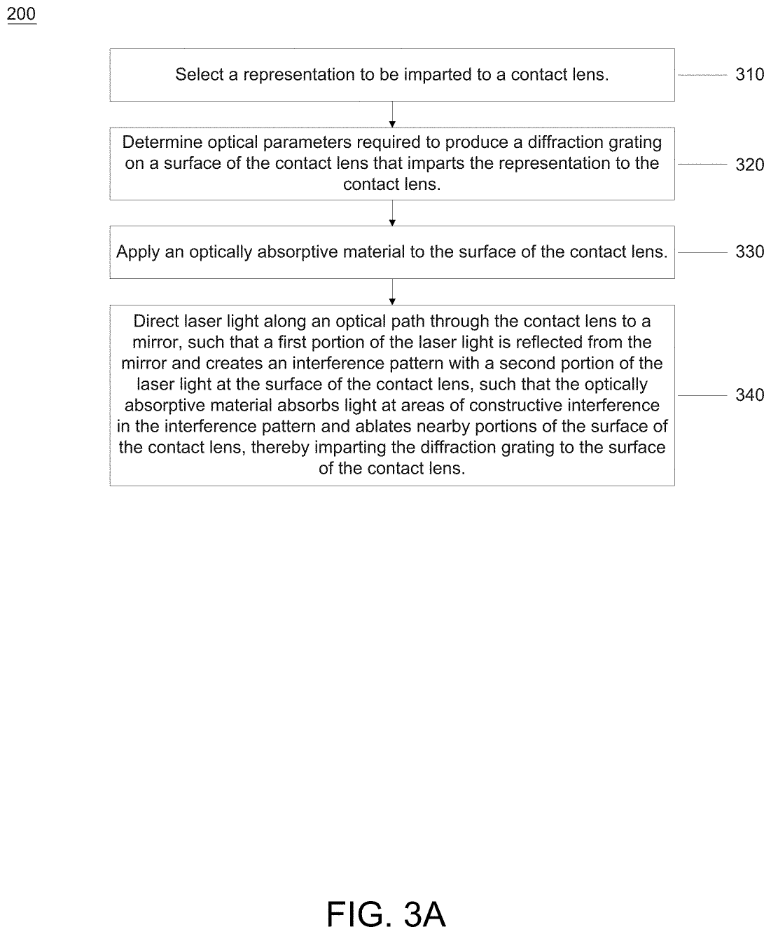

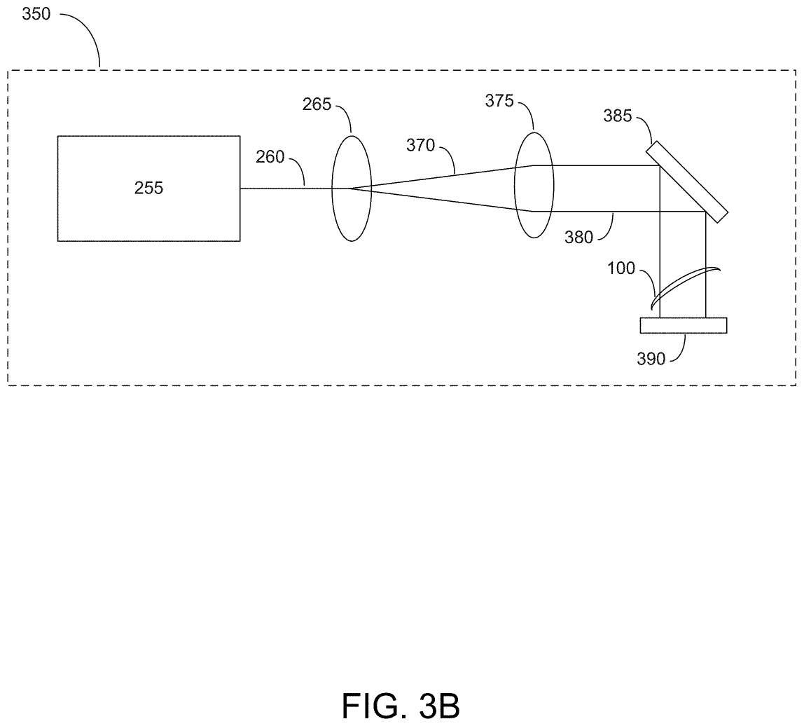

FIG. 3A shows a flowchart for a method 300 of imparting a representation to a wearable ocular device using reflection holography ablation to produce a diffraction grating on a surface of the device. In a first operation 310, the method 300 may comprise selecting a representation to be imparted to the device. The device may be a contact lens. The contact lens may be any contact lens described herein. The device may be bifocals. The device may be an ocular prosthesis.

In a second operation 320, the method 300 may comprise determining optical parameters required to produce a diffraction grating on a surface of the device that imparts the representation to the device. The surface of the device may be a front surface of the contact lens. The surface of the device may be a back surface of the contact lens.

In a third operation 330, the method 300 may comprise applying an optically absorptive material to the surface of the device. The optically absorptive material may be any optically absorptive material described herein, such as any optically absorptive material described herein with respect to FIG. 2A.

In a fourth operation 340, the method 300 may comprise directing laser light along an optical path through the device to a mirror, such that a first portion of the laser light is reflected from the mirror and creates an interference pattern with a second portion of the laser light at the surface of the device, such that the optically absorptive material absorbs light at areas of constructive interference in the interference pattern and ablates nearby portions of the surface of the device, thereby imparting the diffraction grating to the surface of the device. The laser light may be similar to any laser light described herein, such as any laser light described herein with respect to FIG. 2A.

The surface of the device may be configured such that a normal to the surface of the device makes an angle with the laser light. The surface of the device may be configured such that a normal to the surface of the device makes an angle of at least 1 degree, at least 2 degrees, at least 3 degrees, at least 4 degrees, at least 5 degrees, at least 6 degrees, at least 7 degrees, at least 8 degrees, at least 9 degrees, at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 81 degrees, at least 82 degrees, at least 83 degrees, at least 84 degrees, at least 85 degrees, at least 86 degrees, at least 87 degrees, at least 88 degrees, at least 89 degrees, or more, with the laser light. The surface of the device may be configured such that a normal to the surface of the device makes an angle of at most 90 degrees, at most 89 degrees, at most 88 degrees, at most 87 degrees, at most 86 degrees, at most 85 degrees, at most 84 degrees, at most 83 degrees, at most 82 degrees, at most 81 degrees, at most 80 degrees, at most 75 degrees, at most 70 degrees, at most 65 degrees, at most 60 degrees, at most 55 degrees, at most 50 degrees, at most 45 degrees, at most 40 degrees, at most 35 degrees, at most 30 degrees, at most 25 degrees, at most 20 degrees, at most 15 degrees, at most 10 degrees, at most 9 degrees, at most 8 degrees, at most 7 degrees, at most 6 degrees, at most 5 degrees, at most 4 degrees, at most 3 degrees, at most 2 degrees, at most 1 degrees, or less, with the laser light. The surface of the device may be configured such that a normal to the surface of the device makes an angle that is within a range of any two of the preceding values, with the laser light.

The optical path may comprise a spatial filter. The spatial filter may comprise a lens.

The method 300 may be used to impart any representation described herein (such as any representation described herein with respect to FIG. 1A, 1B, 1C, or 1D) to the device. The representation may be an expression or designation.

The expression or designation may be a geometric object. For instance, the diffraction grating may cause a viewer of the device to perceive one or more dots, lines, shapes (such as one or more triangles, quadrilaterals, rectangles, squares, pentagons, hexagons, heptagons, octagons, nonagons, decagons, undecagons, dodecagons, polygons with more than 12 sides, ellipses, ovals, circles, or any other geometric shape). Such markings may represent an indication of one or more optically relevant parameters of the device, such as whether the device is properly centered or oriented on a wearer of the device. In some cases, the markings may represent an indication of whether a contact lens is properly centered or oriented on an eye of a wearer of the contact lens. For instance, the marking may comprise a bump or lenticular that indicates an orientation of the contact lens.

The expression or designation may be a repository of information. For instance, the diffraction grating may cause a viewer of the device to perceiver a barcode, a QR code, or a QR code with a circular hole in its center. The repository of information may be useful for quality control or other tracking purposes. For instance, the repository of information may enable tracking of the device during manufacturing or during an ophthalmological study or clinical trial.

The expression or designation may be a character or term. The character or term may be a character or term selected from any language, such as Mandarin, Spanish, English, Hindi, Arabic, Portuguese, Bengali, Russian, Japanese, Punjabi, German, Javanese, Wu, Malay, Telugu, Vietnamese, Korean, French, Marathi, Tamil, Urdu, Turkish, Italian, Yue, Cantonese, Thai, Gujarati, Jin, Min, Persian, Polish, Pashto, Kannada, Xiang, Malayalam, Sundanese, Hausa, Odia, Burmese, Hakka, Ukrainian, Bhojpuri, Tagalog, Yoruba, Maithili, Uzbek, Sindhi, Amharic, Fula, Romanian, Oromo, Igbo, Azerbaijani, Awadhi, Gan, Cebuano, Dutch, Kurdish, Serbo-Croatian, Malagasy, Saraiki, Nepali, Sinhalese, Chittagonian, Zhuang, Khmer, Turkmen, Assamese, Madurese, Somali, Marwari, Magahi, Haryanvi, Hungarian, Chhattisgarhi, Greek, Chewa, Deccan, Akan, Kazakh, Sylheti, Zulu, Czech, Kinyarwanda, Dhundhari, Haitian, Creole, Ilocano, Quechua, Kirundi, Swedish, Hmong, Shona, Uyghur, Hiligaynon, Ilonggo, Mossi, Xhosa, Belarusian, Balochi, Konkani, or any other language.

The expression or designation may be an image, such as one or more logos, brands, photographs, works of art, cartoons, or other images. The image may be obtained through an image scanning procedure.

The expression or designation may be configured to alter an appearance of an eye of a wearer of the device for artistic purposes, such as for use in movies or other live action performances. In some cases, the expression or designation may be configured to alter an appearance of an eye of a wearer of a contact lens for artistic purposes. For instance, the expression or designation may alter the appearance of the wearer's eye such that the wearer appears to have the eyes of an animal, monster, or other non-human.

The expression or designation may be a color.

The method 300 may further comprise repeating any 1, 2, 3, or 4 of operations 310, 320, 330, and 340 to impart a plurality of diffraction gratings to the surface of the device. The method 300 may further comprise repeating any 1, 2, 3, or 4 of operations 310, 320, 330, and 340 at least 1 time, at least 2 times, at least 3 times, at least 4 times, at least 5 times, at least 6 times, at least 7 times, at least 8 times, at least 9 times, at least 10 times, or more, to impart at least 1, at least 2, at least 3, at least 4, at least 5, at least 6, at least 7, at least 8, at least 9, at least 10, or more diffraction gratings to the surface of the device. The method 300 may further comprise repeating any 1, 2, 3, or 4 of operations 310, 320, 330, and 340 at most 10 times, at most 9 times, at most 8 times, at most 7 times, at most 6 times, at most 5 times, at most 4 times, at most 3 times, at most 2 times, or less, to impart at most 10, at most 9, at most 8, at most 7, at most 6, at most 5, at most 4, at most 3, at most 2, or fewer diffraction gratings to the surface of the device. The method 300 may further comprise repeating any 1, 2, 3, or 4 of operations 310, 320, 330, and 340 a number of times that is within a range defined by any two of the preceding values to impart a number of diffraction gratings that is within a range defined by any two of the preceding values to the surface of the device.

For instance, the method 300 may further comprise repeating any 1, 2, 3, or 4 of operations 310, 320, 330, and 340 a total of three times to impart first, second, and third diffraction gratings to the surface of the device. The first diffraction grating may impart a red hue to the device. The second diffraction grating may impart a green hue to the device. The third diffraction grating may impart a blue hue to the device. The red, green, and blue hues may be chosen to impart a desired color to the device. The desired color may be chosen from a color chart, such as any color chart described herein. For instance, the desired color may be chosen from a CIE color chart such as that described herein with respect to FIG. 1C or a condensed CIE color chart such as that described herein with respect to FIG. 1D.

The method 300 may further comprise, prior to operation 310, (i) selecting a desired color to be imparted to the device, and (ii) determining optical parameters required to produce the first, second, and third diffraction gratings.

The method 300 may further comprise, prior to operation 310, (i) using an optical spectrometer or digital camera to determine a desired color to be imparted to the device, and (ii) determining optical parameters required to produce the first, second, and third diffraction gratings.

The expression or designation may be an artificial pupil. The artificial pupil may comprise a moth eye structure.

The method 300 may further comprise removing the optically absorptive material from the surface of the device.

FIG. 3B shows an optical setup 350 for reflection holography ablation of a wearable ocular device. The optical setup 350 may be used to enact the method 300. The optical setup 350 may comprise a laser 255. The laser may be similar to any laser described herein. For instance, the laser may be similar to any laser described herein with respect to FIG. 2A. The laser may emit laser light 360. The laser light 260 may be similar to any laser light described herein. For instance, the laser light 260 may be similar to any first laser light described herein with respect to FIG. 2A. The laser light may be directed to an optical filter 265. The optical filter may comprise a lens. The optical filter may expand the laser light and direct expanded laser light 370 along an optical path to a collimating lens 375. The collimating lens may direct collimated laser light 380 to a mirror 385, which may reflect the collimated laser light through device 100 to mirror 390. Mirror 390 may reflect the collimated laser light, producing an interference pattern at the surface of device 100.

Wearable ocular devices of the present disclosure (such as wearable ocular device 100 described herein) may be processed using variations of the holography ablation methods (such as methods 200 and 300 described herein). For instance, the devices may be processed using edge-lit holography.

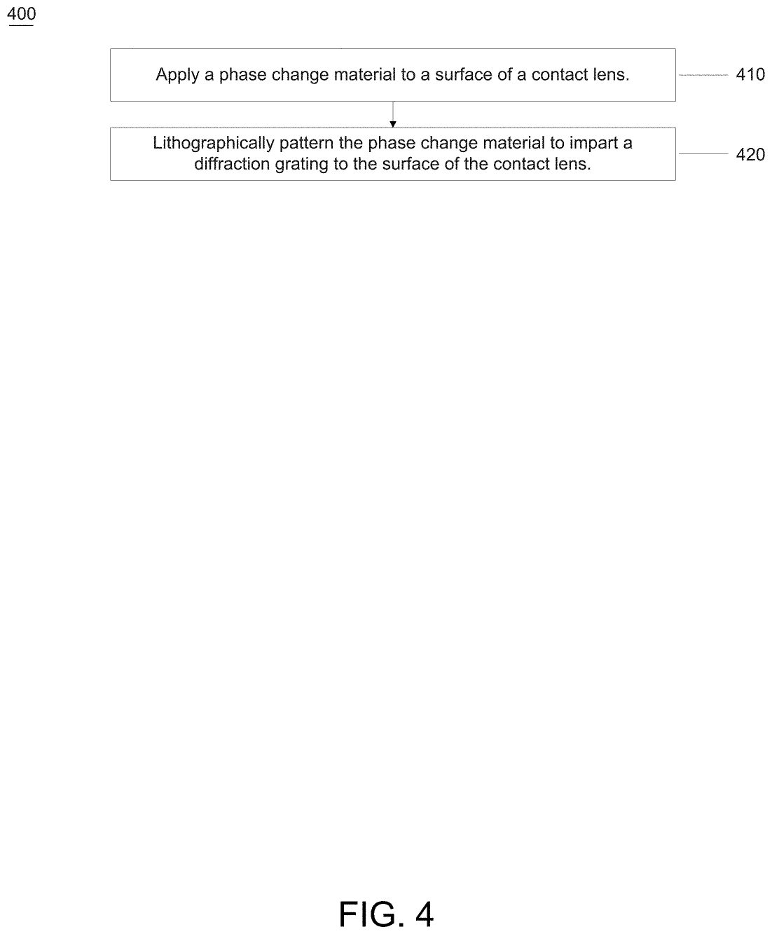

FIG. 4 shows a flowchart for a method 400 of imparting a representation to a wearable ocular device using a phase change material applied to a surface of the device to produce a diffraction grating on the surface of the device. In a first operation 410, the method 400 may comprise applying a phase change material to a surface of the device. The device may be a contact lens. The contact lens may be any contact lens described herein. The device may be bifocals. The device may be an ocular prosthesis. The phase change material may be a photopolymer. The surface of the device may be a front surface of the contact lens. The surface of the device may be a back surface of the contact lens. The photopolymer may be a thin film. The photopolymer may be a thin film. The photopolymer may have a thickness of at least 1 nm, at least 2 nm, at least 3 nm, at least 4 nm, at least 5 nm, at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, at least 200 nm, at least 300 nm, at least 400 nm, at least 500 nm, at least 600 nm, at least 700 nm, at least 800 nm, at least 900 nm, at least 1 μm, at least 2 μm, at least 3 μm, at least 4 μm, at least 5 μm, at least 6 μm, at least 7 μm, at least 8 μm, at least 9 μm, at least 10 μm, at least 20 μm, at least 30 μm, at least 40 μm, at least 50 μm, at least 60 μm, at least 70 μm, at least 80 μm, at least 90 μm, at least 100 μm, at least 200 μm, at least 300 μm, at least 400 μm, at least 500 μm, at least 600 μm, at least 700 μm, at least 800 μm, at least 900 μm, or at least 1,000 μm, or more. The photopolymer may have a thickness of at most 1,000 μm, at most 900 μm, at most 800 μm, at most 700 μm, at most 600 μm, at most 500 μm, at most 400 μm, at most 300 μm, at most 200 μm, at most 100 μm, at most 90 μm, at most 80 μm, at most 70 μm, at most 60 μm, at most 50 μm, at most 40 μm, at most 30 μm, at most 20 μm, at most 10 μm, at most 9 μm, at most 8 μm, at most 7 μm, at most 6 μm, at most 5 μm, at most 4 μm, at most 3 μm, at most 2 μm, at most 1 μm, at most 900 nm, at most 800 nm, at most 700 nm, at most 600 nm, at most 500 nm, at most 400 nm, at most 300 nm, at most 200 nm, at most 100 nm, at most 90 nm, at most 80 nm, at most 70 nm, at most 60 nm, at most 50 nm, at most 40 nm, at most 30 nm, at most 20 nm, at most 10 nm, at most 9 nm, at most 8 nm, at most 7 nm, at most 6 nm, at most 5 nm, at most 4 nm, at most 3 nm, at most 2 nm, at most 1 nm, or less. The photopolymer may have a thickness that is within a range defined by any two of the preceding values.

In a second operation 420, the method 400 may comprise lithographically patterning the phase change material to impart a diffraction grating to the surface of the device. For instance, the method 400 may comprise lithographically patterning the photopolymer to impart a diffraction grating to the surface of the device. The method 400 may comprise a variety of lithographic techniques. For instance, the method 400 may comprise exposing the phase change material to exposure light through a photomask. Regions of the phase change material that receive exposure light through the photomask may display a different optical index of refraction from regions of the phase change material that do not receive exposure light through the photomask. Thus, selecting a proper photomask may allow the production of a diffraction grating in the phase change material. The exposure light may comprise UV, deep UV, or extreme UV light. The exposure light may comprise a wavelength of at least 1 nm, at least 2 nm, at least 3 nm, at least 4 nm, at least 5 nm, at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, at least 200 nm, at least 300 nm, or more. The exposure light may comprise a wavelength of at most 300 nm, at most 200 nm, at most 100 nm, at most 90 nm, at most 80 nm, at most 70 nm, at most 60 nm, at most 50 nm, at most 40 nm, at most 30 nm, at most 20 nm, at most 10 nm, at most 9 nm, at most 8 nm, at most 7 nm, at most 6 nm, at most 5 nm, at most 4 nm, at most 3 nm, at most 2 nm, at most 1 nm, or less. The exposure light may comprise a wavelength that is within a range defined by any two of the preceding values. The exposure light may be emitted by a non-laser light source, such as a flood lamp. The exposure light may be emitted by a laser light source, such as any laser light source described herein with respect to FIG. 2A. Alternatively or in combination, the method 400 may comprise subjecting the phase change material to electron-beam lithography, imprint lithography, microimprint lithography, or nanoimprint lithography.

Operation 410 may occur prior to operation 420.

Operation 420 may occur prior to operation 410.

The method 400 may be used to impart any representation described herein (such as any representation described herein with respect to FIG. 1A, 1B, 1C, or 1D) to the device. The representation may be an expression or designation.

The expression or designation may be a geometric object. For instance, the diffraction grating may cause a viewer of the device to perceive one or more dots, lines, shapes (such as one or more triangles, quadrilaterals, rectangles, squares, pentagons, hexagons, heptagons, octagons, nonagons, decagons, undecagons, dodecagons, polygons with more than 12 sides, ellipses, ovals, circles, or any other geometric shape). Such markings may represent an indication of one or more optically relevant parameters of the device, such as whether the device is properly centered or oriented on a wearer of the contact lens. In some cases, the markings may represent an indication of whether a contact lens is properly centered or oriented on an eye of a wearer of the contact lens. For instance, the marking may comprise a bump or lenticular that indicates an orientation of the contact lens.

The expression or designation may be a repository of information. For instance, the diffraction grating may cause a viewer of the device to perceiver a barcode, a QR code, or a QR code with a circular hole in its center. The repository of information may be useful for quality control or other tracking purposes. For instance, the repository of information may enable tracking of the device during manufacturing or during an ophthalmological study or clinical trial.

The expression or designation may be a character or term. The character or term may be a character or term selected from any language, such as Mandarin, Spanish, English, Hindi, Arabic, Portuguese, Bengali, Russian, Japanese, Punjabi, German, Javanese, Wu, Malay, Telugu, Vietnamese, Korean, French, Marathi, Tamil, Urdu, Turkish, Italian, Yue, Cantonese, Thai, Gujarati, Jin, Min, Persian, Polish, Pashto, Kannada, Xiang, Malayalam, Sundanese, Hausa, Odia, Burmese, Hakka, Ukrainian, Bhojpuri, Tagalog, Yoruba, Maithili, Uzbek, Sindhi, Amharic, Fula, Romanian, Oromo, Igbo, Azerbaijani, Awadhi, Gan, Cebuano, Dutch, Kurdish, Serbo-Croatian, Malagasy, Saraiki, Nepali, Sinhalese, Chittagonian, Zhuang, Khmer, Turkmen, Assamese, Madurese, Somali, Marwari, Magahi, Haryanvi, Hungarian, Chhattisgarhi, Greek, Chewa, Deccan, Akan, Kazakh, Sylheti, Zulu, Czech, Kinyarwanda, Dhundhari, Haitian, Creole, Ilocano, Quechua, Kirundi, Swedish, Hmong, Shona, Uyghur, Hiligaynon, Ilonggo, Mossi, Xhosa, Belarusian, Balochi, Konkani, or any other language.

The expression or designation may be an image, such as one or more logos, brands, photographs, works of art, cartoons, or other images. The image may be obtained through an image scanning procedure.

The expression or designation may be configured to alter an appearance of a wearer of the device for artistic purposes, such as for use in movies or other live action performances. In some cases, the expression or designation may be configured to alter an appearance of an eye of a wearer of a contact lens for artistic purposes. For instance, the expression or designation may alter the appearance of the wearer's eye such that the wearer appears to have the eyes of an animal, monster, or other non-human.

The expression or designation may be a color.

The method 400 may further comprise repeating any 1 or 2 of operations 410 and 420 to impart a plurality of diffraction gratings to the surface of the device. The method 400 may further comprise repeating any 1 or 2 of operations 410 and 420 at least 1 time, at least 2 times, at least 3 times, at least 4 times, at least 5 times, at least 6 times, at least 7 times, at least 8 times, at least 9 times, at least 10 times, or more, to impart at least 1, at least 2, at least 3, at least 4, at least 5, at least 6, at least 7, at least 8, at least 9, at least 10, or more diffraction gratings to the surface of the device. The method 400 may further comprise repeating any 1 or 2 of operations 410 and 420 at most 10 times, at most 9 times, at most 8 times, at most 7 times, at most 6 times, at most 5 times, at most 4 times, at most 3 times, at most 2 times, or less, to impart at most 10, at most 9, at most 8, at most 7, at most 6, at most 5, at most 4, at most 3, at most 2, or fewer diffraction gratings to the surface of the device. The method 400 may further comprise repeating any 1 or 2 of operations 410 and 420 a number of times that is within a range defined by any two of the preceding values to impart a number of diffraction gratings that is within a range defined by any two of the preceding values to the surface of the device.

For instance, the method 400 may further comprise repeating any 1 or 2 of operations 410 and 420 a total of three times to impart first, second, and third diffraction gratings to the surface of the device. The first diffraction grating may impart a red hue to the device. The second diffraction grating may impart a green hue to the device. The third diffraction grating may impart a blue hue to the device. The red, green, and blue hues may be chosen to impart a desired color to the device. The desired color may be chosen from a color chart, such as any color chart described herein. For instance, the desired color may be chosen from a CIE color chart such as that described herein with respect to FIG. 1C or a condensed CIE color chart such as that described herein with respect to FIG. 1D.

The method 400 may further comprise, prior to operation 410 or 420, (i) selecting a desired color to be imparted to the device, and (ii) determining optical parameters required to produce the first, second, and third diffraction gratings.

The method 400 may further comprise, prior to operation 410 or 420, (i) using an optical spectrometer or digital camera to determine a desired color to be imparted to the device, and (ii) determining optical parameters required to produce the first, second, and third diffraction gratings.

The expression or designation may be an artificial pupil. The artificial pupil may comprise a moth eye structure.

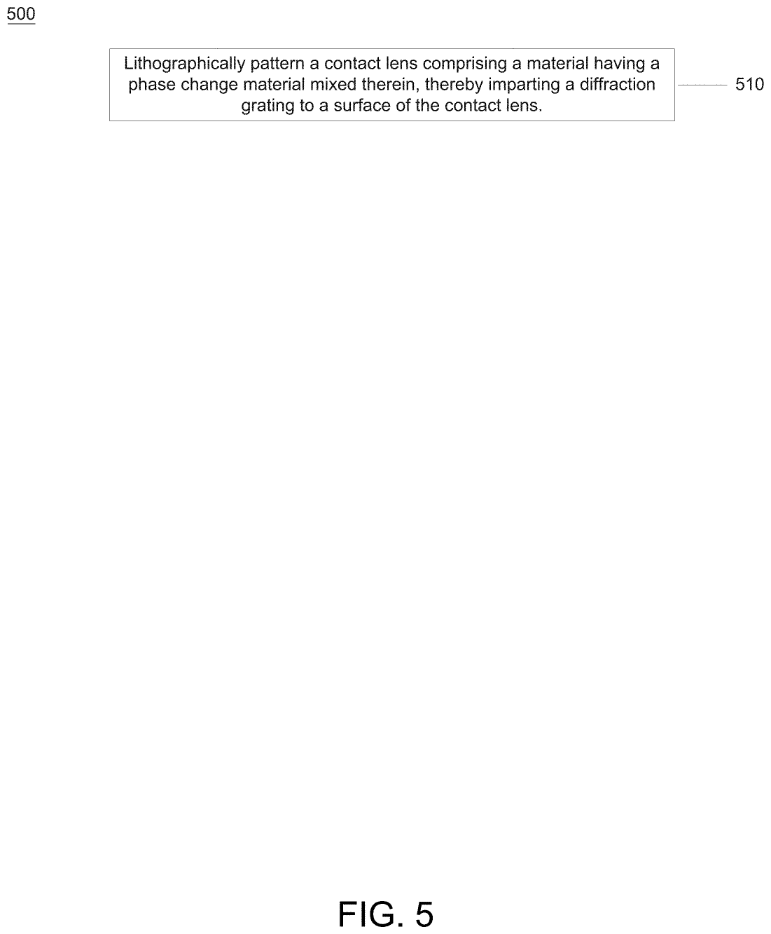

FIG. 5 shows a flowchart for a method 500 of imparting a representation to a wearable ocular device using a phase change material mixed into the device to produce a diffraction grating on a surface of the device. In a first operation 510, the method 500 may comprise lithographically patterning a device comprising a material having a phase change material mixed therein, thereby imparting a diffraction grating to a surface of the device. The device may be any device described herein. The device may be a contact lens. The surface of the device may be a front surface of the contact lens. The surface of the device may be a back surface of the contact lens. The device may be bifocals. The device may be an ocular prosthesis. The phase change material may comprise any phase change material described herein, such as any phase change material described herein with respect to FIG. 4. The method 500 may comprise any lithographic technique described herein, such as any lithographic technique described herein with respect to FIG. 4.

The method 500 may be used to impart any representation described herein (such as any representation described herein with respect to FIG. 1A, 1B, 1C, or 1D) to the device. The representation may be an expression or designation.

The expression or designation may be a geometric object. For instance, the diffraction grating may cause a viewer of the device to perceive one or more dots, lines, shapes (such as one or more triangles, quadrilaterals, rectangles, squares, pentagons, hexagons, heptagons, octagons, nonagons, decagons, undecagons, dodecagons, polygons with more than 12 sides, ellipses, ovals, circles, or any other geometric shape). Such markings may represent an indication of one or more optically relevant parameters of the device, such as whether the device is properly centered or oriented on a wearer of the device. In some cases, the markings may represent an indication of whether a contact lens is properly centered or oriented on an eye of a wearer of the contact lens. For instance, the marking may comprise a bump or lenticular that indicates an orientation of the contact lens.

The expression or designation may be a repository of information. For instance, the diffraction grating may cause a viewer of the device to perceiver a barcode, a QR code, or a QR code with a circular hole in its center. The repository of information may be useful for quality control or other tracking purposes. For instance, the repository of information may enable tracking of the device during manufacturing or during an ophthalmological study or clinical trial.

The expression or designation may be a character or term. The character or term may be a character or term selected from any language, such as Mandarin, Spanish, English, Hindi, Arabic, Portuguese, Bengali, Russian, Japanese, Punjabi, German, Javanese, Wu, Malay, Telugu, Vietnamese, Korean, French, Marathi, Tamil, Urdu, Turkish, Italian, Yue, Cantonese, Thai, Gujarati, Jin, Min, Persian, Polish, Pashto, Kannada, Xiang, Malayalam, Sundanese, Hausa, Odia, Burmese, Hakka, Ukrainian, Bhojpuri, Tagalog, Yoruba, Maithili, Uzbek, Sindhi, Amharic, Fula, Romanian, Oromo, Igbo, Azerbaijani, Awadhi, Gan, Cebuano, Dutch, Kurdish, Serbo-Croatian, Malagasy, Saraiki, Nepali, Sinhalese, Chittagonian, Zhuang, Khmer, Turkmen, Assamese, Madurese, Somali, Marwari, Magahi, Haryanvi, Hungarian, Chhattisgarhi, Greek, Chewa, Deccan, Akan, Kazakh, Sylheti, Zulu, Czech, Kinyarwanda, Dhundhari, Haitian, Creole, Ilocano, Quechua, Kirundi, Swedish, Hmong, Shona, Uyghur, Hiligaynon, Ilonggo, Mossi, Xhosa, Belarusian, Balochi, Konkani, or any other language.

The expression or designation may be an image, such as one or more logos, brands, photographs, works of art, cartoons, or other images. The image may be obtained through an image scanning procedure.

The expression or designation may be configured to alter an appearance of a wearer of the device for artistic purposes, such as for use in movies or other live action performances. In some cases, the expression or designation may be configured to alter an appearance of an eye of a wearer of a contact lens for artistic purposes. For instance, the expression or designation may alter the appearance of the wearer's eye such that the wearer appears to have the eyes of an animal, monster, or other non-human.

The expression or designation may be a color.

The method 500 may further comprise lithographically patterning the device a plurality of times to impart a plurality of diffraction gratings to the surface of the device. The method 500 may further comprise lithographically patterning the device at least 1 time, at least 2 times, at least 3 times, at least 4 times, at least 5 times, at least 6 times, at least 7 times, at least 8 times, at least 9 times, at least 10 times, or more, to impart at least 1, at least 2, at least 3, at least 4, at least 5, at least 6, at least 7, at least 8, at least 9, at least 10, or more diffraction gratings to the surface of the device. The method 500 may further comprise lithographically patterning the device at most 10 times, at most 9 times, at most 8 times, at most 7 times, at most 6 times, at most 5 times, at most 4 times, at most 3 times, at most 2 times, or less, to impart at most 10, at most 9, at most 8, at most 7, at most 6, at most 5, at most 4, at most 3, at most 2, or fewer diffraction gratings to the surface of the device. The method 500 may further comprise lithographically patterning the device a number of times that is within a range defined by any two of the preceding values to impart a number of diffraction gratings that is within a range defined by any two of the preceding values to the surface of the device.

For instance, the method 500 may further comprise lithographically patterning the device a total of three times to impart first, second, and third diffraction gratings to the surface of the device. The first diffraction grating may impart a red hue to the device. The second diffraction grating may impart a green hue to the device. The third diffraction grating may impart a blue hue to the device. The red, green, and blue hues may be chosen to impart a desired color to the device. The desired color may be chosen from a color chart, such as any color chart described herein. For instance, the desired color may be chosen from a CIE color chart such as that described herein with respect to FIG. 1C or a condensed CIE color chart such as that described herein with respect to FIG. 1D.

The method 500 may further comprise, prior to operation 510, (i) selecting a desired color to be imparted to the device, and (ii) determining optical parameters required to produce the first, second, and third diffraction gratings.

The method 500 may further comprise, prior to operation 510, (i) using an optical spectrometer or digital camera to determine a desired color to be imparted to the device, and (ii) determining optical parameters required to produce the first, second, and third diffraction gratings.

The expression or designation may be an artificial pupil. The artificial pupil may comprise a moth eye structure.

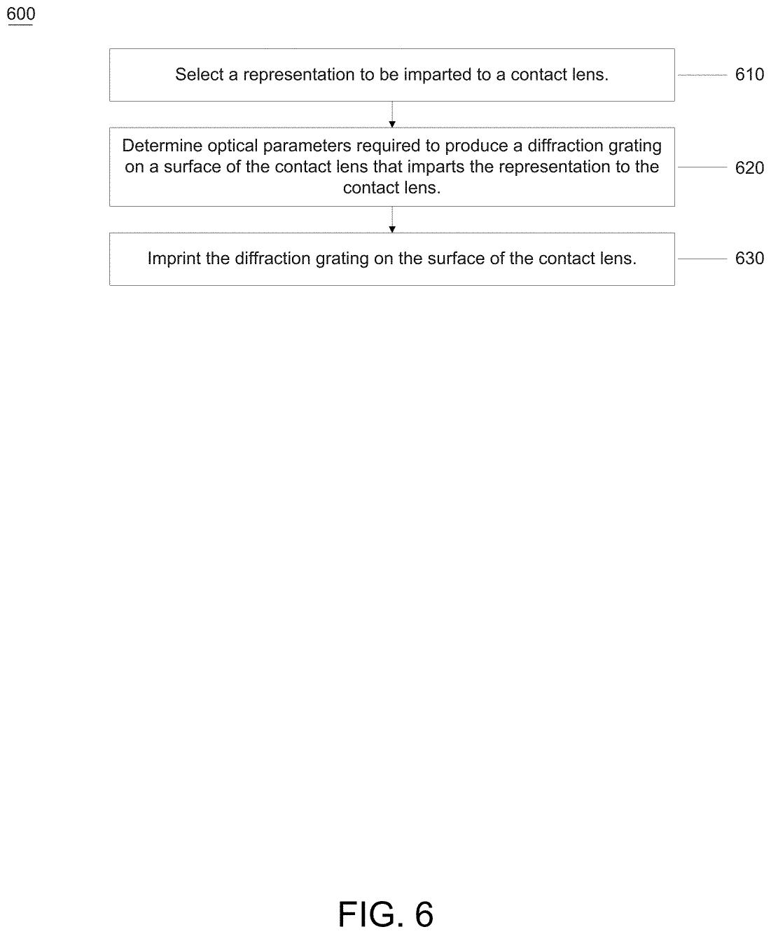

FIG. 6 shows a flowchart for a method 600 of imparting a representation to a wearable ocular device using imprinting to produce a diffraction grating on a surface of the device. In a first operation 610, the method 600 may comprise selecting a representation to be imparted to the device. The device may be a contact lens. The contact lens may be any contact lens described herein. The device may be bifocals. The device may be an ocular prosthesis.

In a second operation 620, the method 600 may comprise determining optical parameters required to produce a diffraction grating on a surface of the device that imparts the representation to the device. The surface of the device may be a front surface of the contact lens. The surface of the device may be a back surface of the contact lens.

In a third operation 630, the method 600 may comprise imprinting the diffraction grating on the surface of the device. Imprinting the diffraction grating on the surface of the device may comprise lithographically patterning and developing a positive or negative photoresist on a substrate. The substrate may comprise silicon, glass, or a metal. The substrate may be flat. The substrate may be curved. The substrate may be concave or convex. A hard material may then be deposited on top of the developed photoresist or in areas of the substrate located that do not contain the developed photoresist. The hard material may comprise a metal, such as nickel or chromium. The hard material may comprise an oxide. The hard material may have a thickness of at thickness of at least 1 nm, at least 2 nm, at least 3 nm, at least 4 nm, at least 5 nm, at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, at least 200 nm, at least 300 nm, at least 400 nm, at least 500 nm, at least 600 nm, at least 700 nm, at least 800 nm, at least 900 nm, at least 1 micrometer (μm), at least 2 μm, at least 3 μm, at least 4 μm, at least 5 μm, at least 6 μm, at least 7 μm, at least 8 μm, at least 9 μm, at least 10 μm, at least 20 μm, at least 30 μm, at least 40 μm, at least 50 μm, at least 60 μm, at least 70 μm, at least 80 μm, at least 90 μm, at least 100 μm, at least 200 μm, at least 300 μm, at least 400 μm, at least 500 μm, at least 600 μm, at least 700 μm, at least 800 μm, at least 900 μm, or at least 1,000 μm, or more. The hard material may have a thickness of at most 1,000 μm, at most 900 μm, at most 800 μm, at most 700 μm, at most 600 μm, at most 500 μm, at most 400 μm, at most 300 μm, at most 200 μm, at most 100 μm, at most 90 μm, at most 80 μm, at most 70 μm, at most 60 μm, at most 50 μm, at most 40 μm, at most 30 μm, at most 20 μm, at most 10 μm, at most 9 μm, at most 8 μm, at most 7 μm, at most 6 μm, at most 5 μm, at most 4 μm, at most 3 μm, at most 2 μm, at most 1 μm, at most 900 nm, at most 800 nm, at most 700 nm, at most 600 nm, at most 500 nm, at most 400 nm, at most 300 nm, at most 200 nm, at most 100 nm, at most 90 nm, at most 80 nm, at most 70 nm, at most 60 nm, at most 50 nm, at most 40 nm, at most 30 nm, at most 20 nm, at most 10 nm, at most 9 nm, at most 8 nm, at most 7 nm, at most 6 nm, at most 5 nm, at most 4 nm, at most 3 nm, at most 2 nm, at most 1 nm, or less. The hard material may have a thickness that is within a range defined by any two of the preceding values. If necessary, the developed photoresist may be removed from the substrate. The substrate and hard material may then serve as a master for imprinting diffraction gratings on a surface of a device. The master may then be imprinted directly into the surface of a device, producing a diffraction grating on a surface of the device. Alternatively or in combination, the substrate and hard material may be used to produce a master (such as a curved nickel master) that may be imprinted into the surface of the device to produce the diffraction grating on the surface of the device.

The master may be manufactured or formed using microfabrication or nanofabrication techniques, such as one or more of solvent cleaning, Piranha cleaning, RCA cleaning, ion implantation, ultraviolet photolithography, deep ultraviolet photolithography, extreme ultraviolet photolithography, electron beam lithography, nanoimprint lithography, wet chemical etching, dry chemical etching, plasma etching, reactive ion etching, deep reactive ion etching, electron beam milling, thermal annealing, thermal oxidation, thin film deposition, chemical vapor deposition, molecular organic chemical deposition, low pressure chemical vapor deposition, plasma enhanced chemical vapor deposition, physical vapor deposition, sputtering, atomic layer deposition, molecular beam epitaxy, electrochemical deposition, wafer bonding, wire bonding, flip chip bonding, thermosonic bonding, wafer dicing, soft lithography, imprint lithography, microimprint lithography, nanoimprint lithography, injection molding, micromilling, three-dimensional printing, or any other suitable microfabrication or nanofabrication manufacturing technique.

The method 600 may be used to impart any representation described herein (such as any representation described herein with respect to FIG. 1A, 1B, 1C, or 1D) to the device. The representation may be an expression or designation.

The expression or designation may be a geometric object. For instance, the diffraction grating may cause a viewer of the device to perceive one or more dots, lines, shapes (such as one or more triangles, quadrilaterals, rectangles, squares, pentagons, hexagons, heptagons, octagons, nonagons, decagons, undecagons, dodecagons, polygons with more than 12 sides, ellipses, ovals, circles, or any other geometric shape). Such markings may represent an indication of one or more optically relevant parameters of the device, such as whether the device is properly centered or oriented on a wearer of the device. In some cases, the markings may represent an indication of whether a contact lens is properly centered or oriented on an eye of a wearer of the contact lens. For instance, the marking may comprise a bump or lenticular that indicates an orientation of the contact lens.

The expression or designation may be a repository of information. For instance, the diffraction grating may cause a viewer of the device to perceiver a barcode, a QR code, or a QR code with a circular hole in its center. The repository of information may be useful for quality control or other tracking purposes. For instance, the repository of information may enable tracking of the device during manufacturing or during an ophthalmological study or clinical trial.

The expression or designation may be a character or term. The character or term may be a character or term selected from any language, such as Mandarin, Spanish, English, Hindi, Arabic, Portuguese, Bengali, Russian, Japanese, Punjabi, German, Javanese, Wu, Malay, Telugu, Vietnamese, Korean, French, Marathi, Tamil, Urdu, Turkish, Italian, Yue, Cantonese, Thai, Gujarati, Jin, Min, Persian, Polish, Pashto, Kannada, Xiang, Malayalam, Sundanese, Hausa, Odia, Burmese, Hakka, Ukrainian, Bhojpuri, Tagalog, Yoruba, Maithili, Uzbek, Sindhi, Amharic, Fula, Romanian, Oromo, Igbo, Azerbaijani, Awadhi, Gan, Cebuano, Dutch, Kurdish, Serbo-Croatian, Malagasy, Saraiki, Nepali, Sinhalese, Chittagonian, Zhuang, Khmer, Turkmen, Assamese, Madurese, Somali, Marwari, Magahi, Haryanvi, Hungarian, Chhattisgarhi, Greek, Chewa, Deccan, Akan, Kazakh, Sylheti, Zulu, Czech, Kinyarwanda, Dhundhari, Haitian, Creole, Ilocano, Quechua, Kirundi, Swedish, Hmong, Shona, Uyghur, Hiligaynon, Ilonggo, Mossi, Xhosa, Belarusian, Balochi, Konkani, or any other language.

The expression or designation may be an image, such as one or more logos, brands, photographs, works of art, cartoons, or other images. The image may be obtained through an image scanning procedure.

The expression or designation may be configured to alter an appearance of a wearer of the device for artistic purposes, such as for use in movies or other live action performances. In some cases, the expression or designation may be configured to alter an appearance of an eye of a wearer of a contact lens for artistic purposes. For instance, the expression or designation may alter the appearance of the wearer's eye such that the wearer appears to have the eyes of an animal, monster, or other non-human.

The expression or designation may be a color.

The method 600 may further comprise repeating any 1, 2, or 3 of operations 610, 620, and 630 to impart a plurality of diffraction gratings to the surface of the device. The method 600 may further comprise repeating any 1, 2, or 3 of operations 610, 620, and 630 at least 1 time, at least 2 times, at least 3 times, at least 4 times, at least 5 times, at least 6 times, at least 7 times, at least 8 times, at least 9 times, at least 10 times, or more, to impart at least 1, at least 2, at least 3, at least 4, at least 5, at least 6, at least 7, at least 8, at least 9, at least 10, or more diffraction gratings to the surface of the device. The method 600 may further comprise repeating any 1, 2, or 3 of operations 610, 620, and 630 at most 10 times, at most 9 times, at most 8 times, at most 7 times, at most 6 times, at most 5 times, at most 4 times, at most 3 times, at most 2 times, or less, to impart at most 10, at most 9, at most 8, at most 7, at most 6, at most 5, at most 4, at most 3, at most 2, or fewer diffraction gratings to the surface of the device. The method 600 may further comprise repeating any 1, 2, or 3 of operations 610, 620, and 630 a number of times that is within a range defined by any two of the preceding values to impart a number of diffraction gratings that is within a range defined by any two of the preceding values to the surface of the device.

For instance, the method 600 may further comprise repeating any 1, 2, or 3 of operations 610, 620, and 630 a total of three times to impart first, second, and third diffraction gratings to the surface of the device. The first diffraction grating may impart a red hue to the device. The second diffraction grating may impart a green hue to the device. The third diffraction grating may impart a blue hue to the device. The red, green, and blue hues may be chosen to impart a desired color to the device. The desired color may be chosen from a color chart, such as any color chart described herein. For instance, the desired color may be chosen from a CIE color chart such as that described herein with respect to FIG. 1C or a condensed CIE color chart such as that described herein with respect to FIG. 1D.

The method 600 may further comprise, prior to operation 610, (i) selecting a desired color to be imparted to the device, and (ii) determining optical parameters required to produce the first, second, and third diffraction gratings.

The method 600 may further comprise, prior to operation 610, (i) using an optical spectrometer or digital camera to determine a desired color to be imparted to the device, and (ii) determining optical parameters required to produce the first, second, and third diffraction gratings.

The expression or designation may be an artificial pupil. The artificial pupil may comprise a moth eye structure.

Method 600 may comprise any operations described in U.S. Pat. No. 7,018,041, entitled “COSMETIC HOLOGRAPHIC OPTICAL DIFFRACTIVE CONTACT LENSES”, filed Jun. 2, 2004, which is herein incorporated by reference in its entirety for all purposes.

Wearable ocular devices of the present disclosure (such as wearable ocular device 100 described herein) may be processed using methods other than the optical and imprinting methods (such as any of methods 200, 300, 400, 500, and 600) described herein. For instance, the devices may be processed using ion beam milling. In such a process, a focused ion beam may be utilized to ablate material from a surface of the device in order to produce any of the devices disclosed herein (such as wearable ocular device 100). Alternatively or in combination, the devices may be processed using semiconductor techniques. For instance, the devices may be processed using chemical etching techniques (such as deep reactive ion etching) to etch material from a surface of the device in order to produce any of the devices disclosed herein (such as wearable ocular device 100). In another example, a device may be subjected to spin coating, lithography, and etching to etch material from a surface of the device in order to produce any of the devices disclosed herein (such as wearable ocular device 100).

Many variations, alterations, and adaptations based on any one or more of the methods 200, 300, 400, 500, and 600 provided herein are possible. For example, the order of the operations of the methods 200, 300, 400, 500, and 600 may be changed, some of the operations removed, some of the operations duplicated, and additional operations added as appropriate. Some of the operations may be performed in succession. Some of the operations may be performed in parallel. Some of the operations may be performed once. Some of the operations may be performed more than once. Some of the operations may comprise sub-operations. Some of the operations may be automated and some of the operations may be manual.

### Computer Systems

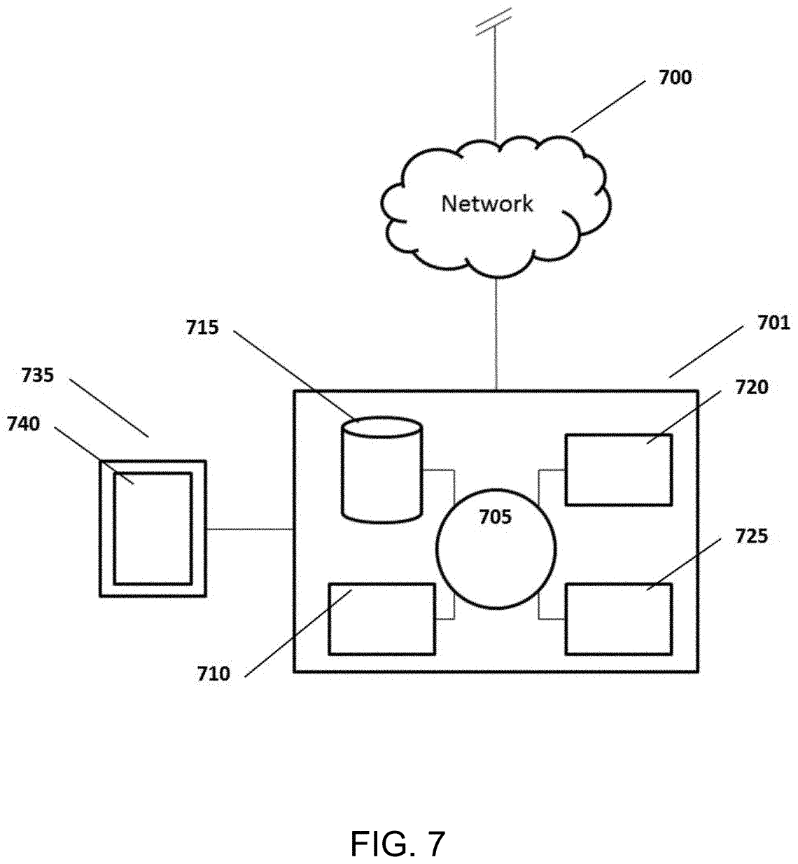

The present disclosure provides computer systems for implementing methods and devices of the present disclosure. FIG. 7 shows a computer system 701 that is programmed or otherwise configured to operate any method or system described herein (such as any method of imparting color to a wearable ocular device described herein). The computer system 701 can regulate various aspects of the present disclosure. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine-readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, enact any of the methods for imparting color to a wearable ocular device as described herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

### Example 1: Contact Lens Quality Control

Systems and methods of the present disclosure may be utilized to provide quality control during manufacturing of contact lenses. For instance, during manufacture of a contact lens, a unique symbol (such as a QR code) may be imparted to the contact lens using the systems and methods described herein (such as the transmission or reflection holographic ablation methods described herein). Each contact lens in a manufacturing process may have a unique QR code imparted to it. Should the contact lens later be found to be defective, the unique QR code may be identified and information about the manufacture of the contact lens may be rapidly attained. For instance, the QR code may be cross-referenced against a database storing manufacturing conditions for the batch and lot in which the contact lens was manufactured. This may allow rapid diagnosis of faults in the manufacturing process and correction of such faults during the manufacture of contact lenses at a future point in time.

### Example 2: Tracking of Contact Lenses During Clinical Trials

Systems and methods of the present disclosure may be utilized to track contact lenses during clinical trials. For instance, a unique symbol (such as a QR code) may be imparted to a contact lens for use in a clinical trial using the systems and methods described herein (such as the transmission or reflection holographic ablation methods described herein). Each contact lens in the clinical trial may have a unique QR code imparted to it. During analysis of the results of the clinical trial, the unique QR code may be identified and information about the progression of the contact lens through the trial may be obtained. For instance, the QR code may be cross-referenced against a database storing information such as any interesting ophthalmological indications presented by a patient wearing the contact lens during the clinical trial. This may provide significant insight into the design of the contact lens during analysis of the results of the clinical trial.

### Example 3: Cosmetic Enhancements for Use in Movies

Systems and methods of the present disclosure may be utilized to provide cosmetic enhancements to the eyes of actors in movies. For instance, a contact lens may be manufactured using the systems and methods described herein (such as the transmission or reflection holographic ablation methods described herein) to create the appearance that a wearer of the contact lens has the eyes of an animal or monster. The contact lens may be worn by an actor during filming of a movie in order to provide a more realistic depiction of the animal or monster.

### Example 4: Determination of Optimal Wavelengths and Angles for Holographic Ablation

Angle calculations were made to determine optimal angles and wavelengths for fabricating the diffraction gratings described herein using the holographic ablation methods described herein. In order to produce the maximum number of colors in a color chart (such as a CIE color chart described herein), red, green, and blue wavelengths must be selected. Based on experience, wavelengths of 640 nm (red), 532 nm (green), and 457 nm (blue) were chosen.

The optical source for ablation should deliver relatively short pulses with relatively high pulse energies and a sufficient coherence length to perform the holographic ablation procedure. Practically, optical sources that meet these requirements emit laser light at one of three wavelengths. 1064 nm lasers have typical coherence lengths of about 60 cm (laser dependent) and typical pulse energies of about 600 mJ (laser dependent). 532 nm lasers have typical coherence lengths of about 30 cm (laser dependent) and typical pulse energies of about 300 mJ (laser dependent). 355 nm lasers have typical coherence lengths of about 15 cm (laser dependent) and typical pulse energies of about 200 mJ (laser dependent). The laser energy may be sufficiently high that that the created interference pattern is above the ablation threshold for the material. The optical source for ablation may be the 1064 light from a Nd:YAG laser or similar. The 532 nm and 355 nm light may be the frequency doubled and tripled light from a Nd:YAG laser or similar.

Based on the red, green, and blue wavelengths chosen, grating spaces can be calculated for different reconstruction angles, as shown in Table 1.

TABLE 1
Grating spacings required to produce selected red, green,
and blue wavelength at different reconstruction angles

| Reconstruction | Grating spacing, | Grating spacing, | Grating spacing, |
|---|---|---|---|
| angle (degrees) | red (microns) | green (microns) | blue (microns) |
| 45 | 0.4525 | 0.3726 | 0.3231 |
| 30 | 0.6400 | 0.5320 | 0.4570 |
| 20 | 0.9356 | 0.7777 | 0.6681 |

Assuming a reference beam normal to the surface of the contact lens, the objective beam angle can be calculated for different reconstruction angles, as shown in Table 2.

TABLE 2
Objective beam angles required to produce selected
red, green, and blue wavelengths at different reconstruction
angles and for different wavelengths

| Construction | | Reconstruction | Objective |
|---|---|---|---|
| wavelength | | angle | beam angle |
| (nm) | Color | (degrees) | (degrees) |
| 1064 | Red | 45 | — |
| 1064 | Green | 45 | — |
| 1064 | Blue | 45 | — |
| 1064 | Red | 30 | 56.2274 |
| 1064 | Green | 30 | 90.0000 |
| 1064 | Blue | 30 | — |
| 1064 | Red | 20 | 34.6532 |
| 1064 | Green | 20 | 43.1602 |
| 1064 | Blue | 20 | 52.7783 |
| 532 | Red | 45 | 35.9998 |
| 532 | Green | 45 | 45.0000 |
| 532 | Blue | 45 | 55.4017 |
| 532 | Red | 30 | 24.5587 |
| 532 | Green | 30 | 30.0000 |
| 532 | Blue | 30 | 35.5953 |
| 532 | Red | 20 | 16.5173 |
| 532 | Green | 20 | 20.000 |
| 532 | Blue | 20 | 23.4626 |
| 355 | Red | 45 | 23.0929 |
| 355 | Green | 45 | 28.1543 |
| 355 | Blue | 45 | 33.3179 |
| 355 | Red | 30 | 16.1017 |
| 355 | Green | 30 | 19.4903 |
| 355 | Blue | 30 | 22.8551 |
| 355 | Red | 20 | 10.9361 |
| 355 | Green | 20 | 13.1928 |
| 355 | Blue | 20 | 15.4075 |

1064 nm light is not able to create the required grating spacings for a 45 degree reconstruction angle and is not able to create the required grating spacing for blue for a 30 degree reconstruction angle. Thus, 1064 nm light may not be desirable for fabricating the gratings described herein.

532 nm light produces reasonable objective beam angles for all reconstruction angles and is less damaging to optics than 355 nm light. Thus, 532 nm light may be optical for fabricating the gratings described herein.

355 nm light produces the smallest objective beam angles. However, 355 nm light may be more difficult to use than 532 nm light because the shorter wavelength light is more damaging to optical coatings.

The maximum allowable objective beam angle may be determined by the numerical aperture (NA) of an objective. A Mitutoyo infinity-corrected long working distance objective was chosen to minimize distortion. Table 3 shows the maximum achievable half angles for a variety of Mitutoyo objectives. The Mitutoyo objectives may be an embodiment of objective 285 described herein with reference to FIG. 2B.

TABLE 3
Maximum achievable half angles for Mitutoyo objectives

| | | Maximum | | Entrance |
|---|---|---|---|---|
| | | half | Standoff | aperture |
| | Numerical | angle | distance | diameter |
| Magnification | Aperture | (degrees) | (mm) | (mm) |
| 2X | 0.055 | 3.153 | 100.0 | 34.0 |
| 5X | 0.14 | 8.048 | 40.0 | 34.0 |
| 10X | 0.28 | 16.260 | 20.0 | 33.5 |
| 20X | 0.42 | 24.835 | 10.0 | 20.0 |
| 50X | 0.55 | 33.367 | 4.0 | 13.0 |
| 100X | 0.80 | 53.130 | 2.0 | 3.0 |
| 50X | 0.42 | 24.835 | 4.0 | 20.5 |
| 100X | 0.55 | 33.367 | 2.0 | 13.0 |

Thus, a variety of Mitutoyo objectives may be selected to create the diffraction gratings, depending on the chosen reconstruction angle.

What is claimed is:

1. A method of imparting a representation to a wearable ocular device, the method comprising:
a. applying an optically absorptive material to a surface of the device;
b. directing a first laser light along a first optical path to the surface of the device;
c. directing a second laser light along a second optical path to the surface of the device; and
d. creating an interference pattern between the first and second laser light at the surface of the device such that the optically absorptive material absorbs light at areas of constructive interference in the interference pattern and ablates nearby portions of the surface of the device, thereby imparting a diffraction grating to the surface of the device.

2. The method of claim 1, wherein the first and second laser light are emitted by a single laser.

3. The method of claim 2, wherein the first and second laser light are directed along the first and second optical paths, respectively, by a spatial filter.

4. The method of claim 3, wherein the first optical path comprises a reference mirror and the second optical path comprises an objective mirror.

5. The method of claim 4, wherein the first laser light is directed from the reference mirror to a first portion of the surface of the device and the second laser light is directed from the objective mirror to a second portion of the surface of the device.

6. The method of claim 5, wherein the first and second portions of the surface of the device partially overlap.

7. The method of claim 1, wherein the representation is an expression or designation.

8. The method of claim 7, wherein the expression or designation is a repository of information about the device.

9. The method of claim 7, wherein the expression or designation is a character or a term.

10. The method of claim 7, wherein the expression or designation is an image.

11. The method of claim 7, wherein the expression or designation is configured to alter an appearance of a wearer of the device for an artistic purpose.

12. The method of claim 7, wherein the expression or designation is a color.

13. The method of claim 1, further comprising repeating (a)-(d) to impart a plurality of diffraction gratings to the surface of the device.

14. The method of claim 13, wherein a first diffraction grating imparts a red hue to the device, a second diffraction grating imparts a green hue to the device, and a third diffraction grating imparts a blue hue to the device.

15. The method of claim 14, further comprising, prior to (a): (i) selecting a desired color to be imparted to the device, and (ii) determining optical parameters required to produce the first, second, and third diffraction gratings.

16. The method of claim 15, further comprising, prior to (a): (i) using an optical spectrometer or digital camera to determine a desired color to be imparted to the device, and (ii) determining optical parameters required to produce the first, second, and third diffraction gratings.

17. The method of claim 1, further comprising removing the optically absorptive material from the surface of the device.

18. The method of claim 1, wherein the device is a contact lens or an ocular prosthesis.

19. A method of imparting a representation to a wearable ocular device, the method comprising:
a. selecting a desired representation to be imparted to the device;
b. determining optical parameters required to produce a diffraction grating on a surface of the device that imparts the desired representation to the device;
c. applying an optically absorptive material to the surface of the device; and
d. directing laser light along an optical path through the device to a mirror, such that a first portion of the laser light is reflected from the mirror and creates an interference pattern with a second portion of the laser light at the surface of the device, such that the optically absorptive material absorbs light at areas of constructive interference in the interference pattern and ablates nearby portions of the surface of the device, thereby imparting the diffraction grating to the surface of the device.

20. A method of imparting a representation to a wearable ocular device, the method comprising:
a. applying a phase change material to a surface of the device; and
b. lithographically patterning the phase change material to impart a diffraction grating to the surface of the device.
